(19) United States
(12) Patent Application Publication
(10) Pub. No.: US 2014/0093596 A1
(43) Pub. Date: Apr. 3, 2014

(54) EXTRACTS FROM PLANTS OF THE TSUGA GENUS AND USES THEREOF IN THE TREATMENT OF INFLAMMATION, IRRITATION AND/OR INFECTION

(71) Applicant: LUCAS MEYER COSMETICS INC., Quebec (CA)

(72) Inventors: Johane GUAY, St. Augustin de Desmaures (CA); Benoit CYR, St. Augustin de Desmaures (CA); Nathalie GENDRON, Neuville (CA); Brigitte PAGE, Quebec City (CA)

(73) Assignee: LUCAS MEYER COSMETICS INC., Quebec (CA)

(21) Appl. No.: 14/047,647

(22) Filed: Oct. 7, 2013

Related U.S. Application Data
(62) Division of application No. 12/936,179, filed on Mar. 29, 2011, now abandoned.
(60) Provisional application No. 61/041,553, filed on Apr. 1, 2008.

(51) Int. Cl. A61K 36/15; A61Q 19/08; A61Q 19/00; A61K 8/97

(52) U.S. Cl. CPC A61K 36/15; A61K 8/97; A61Q 19/08; A61Q 19/005
USPC 424/770

(57) ABSTRACT

Extracts derived from plants of the Tsuga genus having anti-inflammatory and anti [symbol] microbial properties are provided. The Tsuga extracts are suitable for the treatment of inflammation, irritation and/or infection. For example, for the treatment of dermatological conditions with an associated inflammatory component or infection; for combating the irritant or inflammatory effects of other skin treatment compounds; for combating the irritant or inflammatory effects of environmental factors; for treating obesity-related skin problems (for example, inflammation, redness, erythema, rashes and/or bacterial infections caused by skin folds); for combating the irritant or inflammatory effects of, or infection due to, exfoliation, laser treatments or hair removal; for treating irritation, inflammation and/or infection associated with diaper rash; for incorporation into dermatological formulations for sensitive skins, and for providing a preservative effect to dermatological formulations, as well as for ameliorating the dermatological effects of ageing.

### FIELD OF THE INVENTION

The present invention relates to fields of pharmaceuticals and cosmetics and, in particular, to extracts from plants of the Tsuga genus for use to ameliorate inflammation, irritation and/or infection.

### BACKGROUND OF THE INVENTION

The skin performs multiple functions such as protection, barrier, temperature control, excretion and also respiration. It is the main target tissue since it is exposed to all environmental hazards. Not only is the skin subjected to germs, toxic chemicals and hostile environments, it is the only organ directly exposed to ultraviolet light (UV). Over time, physiological changes occur to this organ and lead to a decrease in the functionality of the skin. Changes that occur with ageing, for example, include decrease in thickness, loss of moisture, sagging, loss of elasticity, age spots and wrinkles. Hence, a variety of dermatological conditions may occur as a result of ongoing intrinsic factors (for example, chronological ageing, disease and allergies) and/or exposure to a number of extrinsic factors (such as infection, trauma, radiation, toxins and steroid use).

A number of these dermatological conditions are the result of an inflammatory response or include an inflammation component. Skin irritation is probably one of the most common adverse effects resulting from inflammatory reactions in humans. For example. UV light, allergens, exogenous stress and products found in dermatological formulations such as surfactants are all known to induce inflammatory reactions in the epidermis (Welss T, Toxicology in vitro 18:231, 2004).

The use of retinol and its derivatives in skin care products, for example, has many beneficial effects, however, the concentrations of these compounds that may be used is limited because of the severe local irritation, manifested as mild erythema and stratum corneum peeling of the skin, that the compounds induce (Kim B H, Toxicology Letters 146:65, 2003). Retinol and its derivatives are widely used to treat or ameliorate acne, psoriasis, keratinisation disorders and cutaneous malignancies (Boehm B, Exp Opinion Invest Drugs 4:593, 1995). They are also used in cosmetic formulations to reduce wrinkles (Varani J, J Invest Dermatol Svymp 3:57, 1998) and improve the appearance of cellulite (Kligman A M, J Dermatol Treat 10:119, 1999). AHAs (alpha hydroxy acids) are widely used in facial peeling preparations and in anti-ageing and anti-acne dermatological formulations, are also known to induce irritation after treatment and/or prolonged exposure. Other products such as kojic acid, which are used as whitening agents in skin preparations, have likewise been reported to have highly sensitising effects and may cause irritation at their active concentrations.

Psoriasis is a common chronic, recurrent auto-immune disease of the skin characterized by dry, well-circumscribed, silvery, scaling papules and plaques of various sizes. Psoriasis varies in severity from one or two lesions to widespread dermatosis, sometimes associated with disabling arthritis or exfoliation. Psoriasis is a complex disease; its cause is unknown, but the thick scaling has traditionally been attributed to increased epidermal cell proliferation and concomitant dermal inflammation. Macroscopically, psoriasis is characterized by underlying skin redness (inflammation and accompanying angiogenesis), with overlying keratinocyte hyperproliferation.

Extracts from plants and specific compounds obtained from plant sources are often used in cosmetic and pharmaceutical compositions. For many years in various cultures, medicinal plant extracts have been used for treatment of certain disorders and as cosmetics. For example, Aloe vera promotes a variety of anti-inflammatory responses in the body, reducing swelling from injuries and promoting recovery from infections. Such anti-inflammatory responses not only aid in the relief of pain and discomfort, but also enhance the overall wound healing process. Chamomile is known to improve tissue regeneration, reduce inflammation and encourage the healing of wounds. Flavonoids such as apigenin as well as a distinctive blue essential oil (azulene) derived from chamomile have been found to reduce inflammation and encourage the healing of wounds. Salix alba (willow bark) extract is a natural source of salicylic acid, which is a well-known anti-inflammatory product.

Topical skin applications are known in the art to help shield the skin from the vagaries of the environment. Conventional skin protection typically attempts to either protect the skin from UV light (see U.S. Pat. No. 5,141,741) or provide additional agents capable of neutralizing free radicals (U.S. Pat. No. 6,764,693). Methods of inhibiting either chronological or photo-ageing of the skin by application of UV blocking compounds in combination with compounds that inhibit MMPs have also been reported (U.S. Pat. Nos. 5,837,224; 6,130,254 and 6,365,630 and U.S. Patent Application Publication No. 20010053347). Mercaptoketone and mercaptoalcohol compounds that inhibit the activity of MMPs and their use in treating or controlling disease states such as arthropathy, dermatological conditions, bone resorption, inflammatory diseases and tumor invasion have also been described (U.S. Pat. No. 6,307,101).

Addition of certain plant extracts or phyto-compounds to preparations, such as lotions, creams and gels, to treat dermatological disorders has also been reported. These cosmetic compositions serve to shield the skin from UV light (U.S. Pat. Nos. 4,857,325; 5,141,741 and 6,342,208) and act as antioxidants in the neutralization of free radicals (U.S. Pat. No. 4,923,697). Some fruit extract-containing dermatological agents, capable of neutralizing free radicals, additionally moisturize and facilitate the hydration of the skin (see U.S. Pat. No. 6,800,292).

Other plant extracts useful in dermo-cosmetics have been described (see U.S. Pat. Nos. 6,682,763; 5,824,320 and 6,406,720). Here, external agents derived from olive plants are reported as having skin-beautifying effects, in particular, an anti-ageing effect related to the prevention and elimination of wrinkles and sags of the skin (U.S. Pat. No. 6,682,763). Furthermore, a whitening effect, which can lighten (U.S. Pat. No. 5,073,545) or prevent dark skin, melasma, ephelis and darkening or dullness of the skin has been reported (U.S. Pat. No. 6,682,763). Plant extracts useful in the treatment of eczema and/or psoriasis (U.S. Pat. Nos. 6,676,975 and 4,855,131), and for maintaining general skin care (U.S. Pat. No. 6,193,975) have also been described.

International Patent Application No. PCT/CA04/02007 (WO 2006/053415) describes a large number of plant extracts that are useful for the preparation of dermatological formulations and uses of these formulations for ameliorating the effects of ageing and for the routine care of skin, hair and/or nails.

The Tsuga genus is a genus of conifers in the family Pinaceae. Plants in this genus are known under the common name of “hemlock.” Catechol tannins extracted from Tsuga or hemlock have been described for the treatment of burns (U.S. Pat. No. 2,276,241; GB Patent No. 544,615 and Canadian Patent No. 406,408) due to their tanning action. As further described in these patents, tannins are not germicidal and as such the burn treatment compositions further comprise an effective germicide, specifically a phenolic compound, which is compatible with the tannin.

Tsuga extracts have been described for their deodorant properties. For example, Japanese Patent Application Publication No. 2002087973 describes extracts from Tsuga as part of cosmetic compositions for suppressing human body odour, Japanese Patent Application Publication No. 4030855 describes a mousse-like deodorant containing several plant extracts including a Tsuga extract, and U.S. Pat. No. 4,898,727 describes a deodorant containing several plant extracts including a Tsuga extract, a filter using same and a method of producing the deodorant.

European Patent Application Publication No. 0 870 507 describes a synergistic anti-bacterial composition that includes an extract of botanical materials and an essential oil. The essential oil is described as having anti-microbial activity, whereas the extract of botanical materials has significantly lower activity, or no anti-microbial activity, when used alone. A variety of potential botanical materials are described in the application including Tsuga, with the preferred material being a combination of Plantago, Hypericum, Echinacea and Propolis.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

It is an object of the invention to provide extracts from plants of the Tsuga genus and uses thereof in the treatment of inflammation, irritation and/or infection. In accordance with one aspect of the present invention, there is provided a use of an extract from a plant of the Tsuga genus to ameliorate skin inflammation, irritation and/or infection.

In accordance with another aspect of the invention, there is provided an extract from a plant of the Tsuga genus, or one or more active ingredients isolated therefrom, for use to ameliorate inflammation, irritation and/or infection in a subject in need thereof.

In accordance with another aspect of the invention, there is provided an extract from a plant of the Tsuga genus for use to treat skin inflammation, irritation and/or infection.

In accordance with another aspect of the invention, there is provided a dermatological formulation comprising an extract from a plant of the Tsuga genus, or one or more active ingredients isolated therefrom, and one or more of retinol, a retinol derivative and an alpha-hydroxy acid.

In accordance with another aspect of the invention, there is provided a use of an extract from a plant of the Tsuga genus, or one or more active ingredients isolated therefrom, in the preparation of a dermatological formulation for ameliorating skin inflammation, irritation and/or infection.

In accordance with another aspect of the invention, there is provided a method of ameliorating skin inflammation, irritation and/or infection comprising topically administering to a subject in need thereof an effective amount of an extract from a plant of the Tsuga genus, or one or more active ingredients isolated therefrom.

### BRIEF DESCRIPTION OF THE FIGURES

These and other features of the invention will become more apparent in the following detailed description in which reference is made to the appended drawings.

FIG. 1 presents a graph illustrating the viability of skin cells treated with different concentrations of a Tsuga canadensis extract in accordance with one embodiment of the invention.

FIG. 2 illustrates the anti-inflammatory effect of a Tsuga canadensis extract in accordance with one embodiment of the invention. The bar graph depicts the inhibition of UVA-induced interleukin-1 (IL-1) release in human keratinocytes in vitro by different concentrations of the Tsuga canadensis extract “207-20156A”.

FIG. 3 presents a photograph of areas of the skin of a human volunteer that have been treated with a Tsuga extract in accordance with one embodiment of the invention and demonstrates the anti-inflammatory effect of the Tsuga canadensis extract on retinol-induced inflammation of the skin in vivo. Area 1 was treated with a cream containing 0.5% (w/w) retinol; Area 2 was treated with a cream containing 0.5% (w/w) retinol and 5% (w/w) of the Tsuga extract; Area 3 was treated with a cream containing 1% (w/w) retinol, and Area 4 was treated with a cream containing 0.5% (w/w) retinol and 5% (w/w) of the Tsuga extract.

### DETAILED DESCRIPTION OF THE INVENTION

The present invention relates to the newly identified anti-inflammatory and anti-microbial properties of extracts derived from plants of the Tsuga genus (“Tsuga extracts”). In its broadest aspect, therefore, the present invention provides for the use of the Tsuga extracts to ameliorate inflammation, irritation and/or infection.

In accordance with one embodiment of the invention, the Tsuga extracts are also capable of inhibiting one or more of angiogenesis, contractile force of fibroblasts and/or UV-induced protease activity. These properties, together with the anti-inflammatory and anti-microbial properties of the Tsuga extracts, render the extracts well-suited for the treatment of dermatological conditions with an associated inflammatory component or infection (such as, for example, psoriasis, rosacea, erythema and acne); for combating the irritant or inflammatory effects of other skin treatment compounds (such as retinol); for combating the irritant or inflammatory effects of environmental factors (such allergens or over-exposure to the sun); for treating obesity-related skin problems (for example, inflammation, redness, erythema, rashes and/or bacterial infections caused by skin folds); for combating the irritant or inflammatory effects of, or infection due to, cosmetic or surgical skin procedures, such as peels, exfoliation, laser treatments, hair removal, plastic surgery and the like; for treating irritation, inflammation and/or infection associated with diaper rash; for incorporation into dermatological formulations for sensitive skins, and for providing a preservative effect to dermatological formulations, as well as for ameliorating the dermatological effects of ageing.

Thus, in one aspect, the present invention provides for the use of Tsuga extracts for treatment of skin inflammation, irritation and/or infection. In another aspect, the invention provides for the use of Tsuga extracts in dermatological formulations in order to combat the irritant or inflammatory effects of other components of the formulation on the skin, for example, to render the formulation more amenable for sensitive skins or to combat the irritant effect of retinol or other irritants or sensitizing agents in skin care preparations, such as anti-ageing or anti-acne formulations. In this context, the Tsuga extract may be included in the skin care preparation or may be applied separately from the skin care preparation. In a further aspect, the invention provides for the use of the Tsuga extract in dermatological formulations for ameliorating the irritant/inflammatory effects of environmental factors, for example in “after sun” formulations. In another aspect, the present invention provides for the use of the Tsuga extract in dermatological formulations for providing a soothing or healing effect to inflamed, irritated or infected skin, for example, resulting from obesity-related skin problems, from exfoliation, hair removal, laser treatments or diaper rash. In another aspect, the invention provides for the use of the Tsuga extract in dermatological formulations in order to provide a preservative effect to the formulation. In a further aspect, the invention provides for the use of the Tsuga extracts as anti-ageing or anti-acne agents.

The ability of the Tsuga extracts to combat the irritant effects of retinol, retinol derivatives and other irritants, such as alpha-hydroxy acids (AHAs) and skin whitening agents, allows for the use of formulations that comprise the extract and higher than standard amounts of these agents. In one embodiment, therefore, the present invention provides for dermatological formulations that comprise a Tsuga extract and one or more of retinol, a retinol derivative, an AHA or a skin whitening agent, wherein the retinol, retinol derivative or AHA is present in a higher than standard amount. In another embodiment, the present invention provides for dermatological formulations that comprise a Tsuga extract and standard amounts of one or more of retinol, a retinol derivative, an AHA or a skin whitening agent. The dermatological compositions comprising a Tsuga extract and retinol, a retinol derivative, or an AHA may be used, for example, to ameliorate the dermal signs of ageing or to treat acne.

### DEFINITIONS

Unless defined otherwise, all technical and scientific terms used herein have the same meaning as commonly understood by one of ordinary skill in the art to which this invention belongs.

The term “plant material,” as used herein, refers to any part or parts of a specified plant taken either individually or in a group. Examples include, but are not limited to, leaves, needles, roots, bark, stems, buds, twigs, cones, branches and the like.

The term “extract,” as used herein with reference to a specified plant, refers to a composition prepared by contacting plant material with a solvent following the procedures described herein. The extract can optionally be subjected to one or more separation and/or purification steps.

The term “isolated,” as used herein in the context of an isolated compound (or active ingredient), refers to a compound that is in an environment different from that in which the compound naturally occurs. “Isolated” is meant to include compounds that are within samples that are substantially enriched for the compound of interest and/or in which the compound of interest is partially or substantially purified.

The term “substantially pure,” as used herein refers to a compound (or active ingredient) that is removed from its natural environment and that constitute at least about 50% of a sample, for example at least about 60%, at least about 70%, at least about 80% or at least about 90% of a sample.

The term “skin cell,” as used herein, refers to a cell normally present within the skin of a mammal and includes, but is not limited to, keratinocytes, fibroblasts, endothelial cells (including vascular endothelial cells), basal cells, granular cells, Merkel cells, melanocytes, Langerhans cells, leukocytes, mastocytes, nerve cells, adipose cells and macrophages.

The term “attenuate,” as used herein, means to reduce or inhibit, wherein the inhibition may be complete or partial inhibition.

The term “cell migration,” as used herein, refers to the movement, typically abnormal, of a cell or cells from one locus to another. Examples of cell migration include the movement of endothelial cells during angiogenesis.

A “dermatological formulation,” as used herein, refers to a pharmaceutical composition or a cosmeceutical composition formulated for topical administration to the skin. In one embodiment, the dermatological formulation is for administration to a portion or portion of the skin affected by a dermatological condition or disorder.

The term “dermatological condition,” as used herein, refers to a condition, such as a disease, disorder, irritation, reaction and the like, present in the skin of a subject that is caused by intrinsic or extrinsic factors and/or by ageing.

The term “ameliorate,” as used herein, means to make more tolerable (for example by reducing the incidence or severity), to heal or to cure.

The term “treatment,” as used herein, refers to an intervention performed with the intention of improving a recipient's status. The improvement can be subjective or objective and is related to the amelioration, either temporary or long-term, of one or more of the symptoms associated with a condition being treated. In some embodiments, treatment includes the prevention of the development of the condition. Thus, in various embodiments, the term treatment includes the prevention (prophylaxis), moderation, reduction, and/or curing of a condition at various stages. In certain embodiments, prevention of deterioration of a recipient's status is also encompassed by the term. Those in need of treatment thus may include those already having the condition as well as those prone to, or at risk of developing, the condition and those in whom the condition is to be prevented.

The term “subject,” as used herein, refers to an individual in need of treatment or who would otherwise benefit from the use of a dermatological formulation in accordance with the invention.

As used herein, the term “about” refers to approximately a +/−10% variation from a given value. It is to be understood that such a variation is always included in any given value provided herein, whether or not it is specifically referred to.

### Tsuga Extracts

The present invention provides for extracts from plants of the Tsuga genus (“Tsuga extracts”) suitable for dermatological use. In accordance with the present invention, the Tsuga extracts have anti-inflammatory and anti-microbial activity. In one embodiment of the invention, the Tsuga extracts are also capable of inhibiting one or more of angiogenesis, contractile force of fibroblasts or activity of UV-induced proteases.

In accordance with the present invention, the Tsuga extracts are solvent-based extracts obtained by solvent extraction of plant material from a selected Tsuga plant. The selected Tsuga plant can be, for example, Tsuga canadensis; Tsuga caroliniana; Tsuga chinensis; Tsuga diversifolia; Tsuga dumosa; Tsuga forrestii; Tsuga heterophylla; Tsuga mertensiana or Tsuga sieboldii. In one embodiment of the present invention, the Tsuga plant is a plant native to North America, i.e. Tsuga canadensis; Tsuga caroliniana; Tsuga heterophylla or Tsuga mertensiana. In another embodiment, the Tsuga plant is a plant native to Asia, i.e. Tsuga chinensis; Tsuga diversifolia; Tsuga dumosa; Tsuga forrestii or Tsuga sieboldii. In another embodiment, the Tsuga plant is Tsuga canadensis; Tsuga heterophylla or Tsuga diversifolia.

The solvent used for the preparation of the extract can be an aqueous solvent (such as water or a buffer), or it can be a liquid organic compound, or a combination of an aqueous solvent and a liquid organic compound. In some embodiments, the solvent may be a supercritical or sub-critical fluid. In one embodiment of the invention, the Tsuga extract is an aqueous, alcoholic or aqueous-alcoholic extract. In another embodiment, the Tsuga extract is an aqueous, glycolic or aqueous-glycolic extract.

### Preparation of the Tsuga Extracts

The Tsuga extracts in accordance with the invention are obtained by solvent extraction of plant material from a selected Tsuga plant.

### Plant Material

The plant material is derived from one or a combination of the species of Tsuga noted above, i.e. Tsuga canadensis; Tsuga caroliniana; Tsuga chinensis; Tsuga diversifolia; Tsuga dumosa; Tsuga forrestii; Tsuga heterophylla; Tsuga mertensiana and/or Tsuga sieboldii. The plant material employed in the extraction process can be the entire plant (tree), or it can be one or more distinct tissues from the plant or plants, for example, leaves (needles), cones, roots, branches, bark, stems, twigs, buds or various combinations thereof. In one embodiment of the invention, the Tsuga extract is prepared from needles, twigs, small branches, bark, or any combination thereof.

The plant material can be fresh, dried or frozen. In one embodiment, the plant material used in the preparation of the Tsuga extracts is dried. The plant material may be used immediately after harvesting or it can be stored for a period of time prior to being subjected to the extraction process. If the plant material is stored, it can be treated prior to storage, for example, by drying, freezing, lyophilizing, or some combination thereof, as is known in the art. The storage time may be of various durations, for example, the storage period may be between a few days and a few years. Typically storage times range between less than one week and about one year in duration.

If desired, the plant material can be treated prior to the extraction process in order to facilitate the extraction process. Typically such treatment results in the plant material being fragmented by some means such that a greater surface area is presented to the solvent. For example, the plant material can be crushed or sliced mechanically, using a grinder or other device to fragment the plant parts into small pieces or particles, or the plant material can be frozen in liquid nitrogen and then crushed or otherwise fragmented into smaller pieces.

If desired and when practicable, the plant material can be derived from a Tsuga plant that was subjected to a stress treatment. A stress treatment comprises contacting or treating the plant, or material from the plant, with one or more stressor with the aim of inducing or eliciting increased production of one or more chemicals. The stressor can be a chemical compound or a physical treatment. Examples of chemical stressors include, but are not limited to, organic and inorganic acids including fatty acids; glycerides; phospholipids; glycolipids; organic solvents; amino acids; peptides; monosaccharides; oligosaccharides; polysaccharides; lipopolysaccharides; phenolics; alkaloids; terpenes; terpenoids; antibiotics; detergents; polyamines; peroxides; ionophores, and the like. Examples of physical stress treatments include, but are not limited to, ultraviolet radiation, sandblasting, low and high temperature stress, and osmotic stress induced by salt or sugars. Nutritional stress is another example of a physical stress and is defined as depriving the plant of essential nutrients (e.g. nitrogen, phosphorus or potassium) in order to induce or elicit increased production of one or more chemicals. The one or more stressor (i.e. chemical compound(s), physical treatment(s), or combination thereof) may be applied continuously or intermittently to the plant material. Various stressors and procedures for stressing plants prior to extract preparation have been described previously (see International Patent Application WO 02/06992) and are suitable for use in accordance with the present invention.

### Solvent Extraction

Various extraction processes are known in the art and can be employed in the process of the present invention (see, for example, International Patent Application WO 02/06992). The solvent extraction process employed in the preparation of the Tsuga extracts typically employs as solvent an aqueous solvent (such as water or a buffer), a liquid organic compound, or a combination thereof. Exemplary liquid organic compounds that can be used as solvents in the extraction process to prepare the Tsuga extracts include, but are not limited to alcoholic solvents, which include primary alcohols such as methyl alcohol (methanol), ethyl alcohol (ethanol), 1-propanol and 1-butanol; secondary alcohols such as 2-propanol and 2-butanol: tertiary alcohols such as 2-methyl-2-propanol, and liquid polyhydric alcohols such as glycerine (glycerol) and glycols. Suitable glycols include, for example, ethylene glycol (1,2-ethandiol), propylene glycol (1,2-propanediol), trimethylene glycol (1,3-propanediol), 1,3-butylene glycol, pentylene glycol (1,2-pentanediol), hexylene glycol (2-methyl-2,4-pentanediol), diethylene glycol, dipropylene glycol and lower molecular weight polyethylene glycols. Other known organic solvents for plant extraction include acetone, tetrahydrofuran, acetonitrile, 1,4-dioxane, pyridine, dimethylsulfoxide, N,N-dimethyl formamide, acetic acid, diethyl ether, hexane, heptane, dichloromethane and ethyl acetate. Supercritical or sub-critical fluids, such as water or carbon dioxide, are also suitable solvents for the preparation of the Tsuga extracts.

In one embodiment of the invention, the solvent employed to prepare the Tsuga extracts comprises an alcohol. In another embodiment, the solvent employed to prepare the Tsuga extracts comprises a primary alcohol or a liquid polyhydric alcohol. In another embodiment, the solvent employed to prepare the Tsuga extracts comprises a supercritical or sub-critical fluid.

When the extraction process is carried out using a solvent that comprises a mixture of an aqueous solvent and a liquid organic compound, the content of the liquid organic compound ranges from about 5% to about 95% by volume. In one embodiment of the invention, the extraction process is carried out using a solvent that comprises a mixture of an aqueous solvent and a liquid organic compound in which the content of the liquid organic compound ranges from about 10% to about 95% by volume. In another embodiment, the extraction process is carried out using a solvent that comprises a mixture of an aqueous solvent and a liquid organic compound in which the content of the liquid organic compound ranges from about 15% to about 95% by volume. In other embodiments, the extraction process is carried out using a solvent that comprises a mixture of an aqueous solvent and a liquid organic compound in which the content of the liquid organic compound ranges from about 10% to about 90% by volume, from 20% to about 95% by volume, from about 20% to about 90% by volume, from about 10% to about 85% by volume and from about 20% to about 85% by volume.

In one embodiment, a solvent that is compatible with mammalian skin is used in the extraction. This can, for example, allow for the extract to be incorporated directly into a dermatological formulation with little, or no, further processing. Examples of such solvents include, but are not limited to, water, an aqueous buffer, a combination of water/buffer with a lower alcohol or an anhydrous lower alcohol. In the context of the present invention, a lower alcohol refers to an alcohol having 1 to 6 carbon atoms, such as a primary, secondary, tertiary or liquid polyhydric alcohol. In one embodiment of the present invention, the solvent for the preparation of the Tsuga extract is selected from water, a lower alcohol or a combination thereof. In another embodiment, the solvent for the preparation of the Tsuga extract comprises a lower alcohol selected from the group of: methyl alcohol (methanol), ethyl alcohol (ethanol), 1-propanol, 1-butanol, 2-propanol, 2-butanol, 2-methyl-1-propanol, 2-methyl-2-propanol, glycerine, ethylene glycol, propylene glycol, diethylene glycol, dipropylene glycol, 1,3-propanediol and 1,3-butylene glycol.

When the extraction process employs a combination of an aqueous solvent and a lower alcohol as solvent, the lower alcohol content of the solvent typically ranges from about 5% to about 95% by volume, for example from about 10% to about 95% by volume. In one embodiment of the invention, the extraction process is carried out using a solvent that comprises a mixture of an aqueous solvent and a lower alcohol in which the content of the lower alcohol ranges from about 15% to about 95% by volume. In another embodiment of the invention, the extraction process is carried out using a solvent that comprises a mixture of an aqueous solvent and a lower alcohol in which the content of the lower alcohol ranges from about 20% to about 95% by volume. In other embodiments, the extraction process is carried out using a solvent that comprises a mixture of an aqueous solvent and a lower alcohol in which the content of the lower alcohol ranges from about 20% to about 90% by volume, and from about 20% to about 85% by volume.

For example, when the extraction process employs a solvent that is an aqueous solvent/primary alcohol mixture, the primary alcohol can be present in an amount between about 20% to about 90% by volume, for example from about 30% to about 90% by volume, whereas when the extraction process employs a solvent that is an aqueous solvent/glycol mixture, the glycol can be present in an amount between about 10% to about 80% by volume, for example from about 10% to about 60% by volume. Similarly, when the extraction process employs a solvent that is an aqueous solvent/glycerine mixture, the glycerine can be present in an amount between about 10% to about 80% by volume, for example from about 10% to about 60% by volume.

In one embodiment, the extraction process employs a solvent that is an aqueous solvent/primary alcohol mixture in which the primary alcohol is present in an amount between about 35% to about 90% by volume. In another embodiment, the extraction process employs a solvent that is an aqueous solvent/primary alcohol mixture in which the primary alcohol is present in an amount between about 40% to about 90% by volume. In another embodiment, the extraction process employs a solvent that is an aqueous solvent/primary alcohol mixture in which the primary alcohol is present in an amount between about 45% to about 90%, between about 45% to about 85%, and between about 50% to about 85% by volume.

In an alternate embodiment, the extraction process employs a solvent that is an aqueous solvent/glycol mixture in which the glycol is present in an amount between about 15% to about 60% by volume. In another embodiment, the extraction process employs a solvent that is an aqueous solvent/glycol mixture in which the glycol is present in an amount between about 15% to about 55% by volume. In another embodiment, the extraction process employs a solvent that is an aqueous solvent/glycol mixture in which the glycol is present in an amount between about 20% to about 55%, and between about 20% to about 50% by volume.

A number of standard extraction techniques known in the art can be employed to prepare the plant extracts. In general, the extraction process entails contacting solid plant material with a solvent with adequate mixing and for a period of time sufficient to ensure adequate exposure of the solid plant material to the solvent such that activity present in the plant material can be taken up by the solvent.

An appropriate amount of the solvent to be used in the extraction can be determined by the skilled worker based on the amount of plant material being employed in the extraction. In one embodiment of the invention, the w/v (g/100 mL) of plant material to solvent used in the extraction process is between about 1/2 and about 1/50. In another embodiment, the w/v (g/100 mL) of plant material to solvent used in the extraction process is between about 1/5 and about 1/50. In another embodiment, the w/v (g/100 mL) of plant material to solvent used in the extraction process is between about 1/10 and about 1/50. In other embodiments, the w/v (g/100 mL) of plant material to solvent used in the extraction process is between about 1/10 and about 1/40; between about 1/10 and about 1/30; and between about 1/10 and about 1/25.

A variety of conditions can be employed for the extraction process. Typically, the extraction procedures are conducted over a period of time between about 10 minutes and about 72 hours at a temperature between about 4° C. and about 50° C. However, temperatures between about 4° C. and about 90° C., for example between about 4° C. and about 70° C. can be employed. Higher temperatures are also contemplated, with or without increased pressure, when certain extraction techniques are employed, for example, pressurised liquid extraction, sub-critical fluid extraction (for example, sub-critical water extraction (SWE)) or supercritical fluid extraction. Similarly, the extraction time may be varied depending on other extraction conditions, such as the solvent and temperature employed, for example, the extraction time can range from several minutes to several days. For example, in one embodiment, the extraction time is at least one hour. In another embodiment, the extraction time is between about one hour and about 72 hours. Determination of appropriate extraction temperatures and times is within the ordinary skills of a worker in the art.

Adequate contact between the solvent and the plant material can be encouraged by shaking, stirring, percolating and/or macerating the suspension. Alternatively, an extraction device equipped with, for instance, a stirring machine, or a soxhlet or other device known in the art can be employed which may improve the extraction efficiency. The extraction can be carried out at ordinary pressure, under pressure or at reduced pressure established by, for example, aspiration. Appropriate extraction conditions can readily be determined or selected by one skilled in the art taking into consideration the production conditions such as production facilities and yields.

In one embodiment, the present invention also provides for the use of supercritical fluid extraction for the preparation of the Tsuga extracts. Supercritical fluid extraction involves the use of a supercritical fluid (SCF) as a solvent. A SCF is a liquid or a gas at atmospheric conditions, but becomes supercritical when it is compressed above its critical pressure and heated above its critical temperature. Supercritical fluids have increased dissolving power in their supercritical regions. A supercritical fluid exhibits properties between those of a gas and a liquid, and has the capacity to dissolve compounds that may only dissolve poorly or not at all in the gas or liquid state. Most components extracted from the plant material, once dissolved, can quickly and cleanly be precipitated or removed from the supercritical fluids by lowering the pressure and/or temperature to achieve separation. Using the method of post-extraction fractionation with a column designed to allow for temperature and pressure drops at different levels to gain the desired results may effect further concentration and purification when desirable.

Supercritical fluid extraction processes are well known in the art, for example, see Martinez, J. L. (Supercritical Fluid Extraction of Nutraceuticals and Bioactive Compounds (2007, CRC Press, Boca Raton, Fla.) and Herrero. M. et al. (2005, Food Chem, 98:136-148).

In general, the starting plant material is placed in an extractor device together with the supercritical fluid, with or without a chemical modifier, at specified pressure and temperature conditions to extract the desired components from the plant material. After extraction, the fluid and the compound are passed through a separator which changes the pressure and temperature, thereby reducing the dissolving power of the supercritical fluid and causing the separation or fractionation of the dissolved components.

Examples of suitable supercritical fluids for the preparation of the Tsuga extracts include water and carbon dioxide. Carbon dioxide has a critical temperature of 31.06° C., a critical pressure of 73.83 bar, and a critical density of 0.460 g/cm³, which allows the use of relatively low temperatures for the extraction process. An exemplary SCF extraction process utilising carbon dioxide as the SCF is as follows. Comminuted plant material is combined with the carbon dioxide with one or more modifiers in an extractor device. The extraction is conducted at a pressure between about 270 to about 320 bar, and a temperature of about 40° C. to about 60° C. The ratio of solvent to starting plant material is typically between about 20:1 and about 80:1 by weight, for example between about 45:1 and about 60:1 by weight.

Preparation of the Tsuga extracts using subcritical fluids, with or without a co-solvent, is also contemplated. Examples of suitable subcritical fluids for preparation of the Tsuga extracts include water and carbon dioxide.

As noted above, in some embodiments, one or more co-solvents (or modifiers) are included in the supercritical fluid or subcritical fluid. Modifiers generally possess volatility between that of the supercritical or subcritical fluid and of the components being extracted, and must be miscible with the supercritical/subcritical fluid. Suitable modifiers include, for example, ethanol, methanol, propanol, acetone, ethyl acetate, methylene chloride, and the like. Water is also a suitable modifier when the supercritical/subcritical fluid is carbon dioxide. Ethanol, for example, can be used as a modifier in a ratio of 35 to 75 kg ethanol solvent per kg of plant material.

Following a typical extraction process, whether using standard pressure or sub- or supercritical fluids), the liquid fraction (the Tsuga extract) can be separated from the solid (insoluble) matter. Separation of the liquid and solid fractions can be achieved by one or more standard separation processes known to those skilled in the art, such as various centrifugation or filtration processes. In one embodiment of the invention, the Tsuga extract is separated from solid matter after the extraction by one or more filtration steps. In another embodiment, the Tsuga extract is separated from solid matter after the extraction by a series of filtration steps.

Once the Tsuga extract has been isolated, its activity can be tested directly or after being diluted in a suitable solvent, or it may be subjected to further procedures. For example, the Tsuga extract can be subjected to one or more additional steps to further purify or concentrate the extract. For example, the extract may be subjected to solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, and the like) with or without high pressure, lyophilization, evaporation, precipitation with various “carriers” (including PVPP, carbon, antibodies, and the like), the use of supercritical fluids (such as CO₂), or various combinations thereof. In one embodiment, the Tsuga extract is subjected to procedures to remove fatty acids or chlorophyll components that may interfere with its activity. Various procedures known in the art may be employed. In one embodiment, one or more additional partitioning steps using an organic solvent, such as hexane, heptane or ethyl acetate, are included. The liquid Tsuga extract can be concentrated and solubilised in an appropriate solvent prior to the one or more partitioning steps, if desired.

In one embodiment of the present invention the Tsuga material is subjected to an extraction process that entails contacting the solid plant material with a solvent with adequate mixing over a period of time between about 10 minutes and about 72 hours at a temperature between about 4° C. and about 50° C. The liquid fraction (the Tsuga extract) is then separated from the solid (insoluble) matter by one or more standard processes known to those skilled in the art.

In one embodiment of the invention, the Tsuga extract is prepared by extracting plant material with an alcoholic solvent alone or in combination with an aqueous co-solvent for a time period between about 4 hours and about 48 hours, for example between about 4 hours and about 24 hours, at a temperature between about 4° C. to about 32° C., for example between about 4° C. to about 25° C. In one embodiment, the Tsuga extract is prepared by extracting plant material using a combination of ethanol and water as the solvent, wherein the range of ethanol:water is between about 50:50 and about 85:15, and wherein the extraction is conducted for a time period between about 4 hours and about 48 hours, for example between about 4 hours and about 24 hours, at a temperature between about 4° C. to about 32° C., for example between about 4° C. to about 25° C. In another embodiment, the Tsuga extract is prepared by extracting plant material using a combination of a glycol and water as the solvent, wherein the range of glycol:water is between about 100:0 and about 20:80, and wherein the extraction is conducted for a time period between about 4 hours and about 48 hours, for example between about 4 hours and about 24 hours, at a temperature between about 4° C. to about 32° C., for example between about 4° C. to about 25° C.

The present invention contemplates that the extraction process and any subsequent purification steps may be carried out on various scales including known large, medium and small-scale methods of preparing extracts.

In one embodiment of the invention, the Tsuga extract is prepared on a large-scale. For example, the Tsuga extract can be prepared on a commercial scale by using the extraction process employed in the initial analytical scale preparation of the extract. The small-scale extraction procedure is simply scaled-up and additional steps of quality control can be included to ensure reproducible results.

Also contemplated are modifications to the small-scale procedure as may be required during scale-up for industrial level production of the Tsuga extract. Such modifications include, for example, alterations to the solvent being used or to the extraction procedure employed in order to compensate for variations that occur during scale-up and render the overall procedure more amenable to industrial scale production, or more cost effective. Modifications of this type are standard in the industry and would be readily apparent to those skilled in the art.

Fractionation and/or Isolation of Active Ingredients

The present invention also provides for purified or semi-purified active ingredients isolated from the Tsuga extracts. In the context of the present invention an “active ingredient” is a compound that is present in the Tsuga extract and has anti-inflammatory and/or anti-microbial properties.

There are a number of techniques well known in the art for isolating active ingredients from plant extracts. For example, purification, partial purification, and/or fractionation can be performed using solid-liquid extraction, liquid-liquid extraction, solid-phase extraction (SPE), membrane filtration, ultrafiltration, dialysis, electrophoresis, solvent concentration, centrifugation, ultracentrifugation, liquid or gas phase chromatography (including size exclusion, affinity, etc.) with or without high pressure, lyophilisation, evaporation, precipitation with various “carriers” (including PVPP, carbon, antibodies, etc.), or various combinations thereof.

Thus in one embodiment of the invention, the Tsuga extract is subjected to one or more of the above techniques, in a sequential fashion, in order to obtain therefrom a isolated active ingredient, or combination of active ingredients, that retains the activity of interest (i.e. has anti-inflammatory and/or anti-microbial activity). Isolated active ingredients can be tested for this activity according to one or more of the procedures described below. Furthermore, and where identification and/or quantification of the isolated active ingredient(s) is desired, analytical techniques including, but not limited to, NMR, GC-MS, TLC, spectrophotometry, microspray. X-ray diffraction and elemental analysis may be performed to characterise the active ingredient(s).

### Testing the Tsuga Extracts

The Tsuga extracts in accordance with the present invention have anti-inflammatory and anti-microbial activity. In one embodiment of the invention, the extracts are additionally capable of inhibiting one or more of angiogenesis, contractile force of fibroblasts or UV-induced protease activity. These properties can be assessed using standard techniques known in the art. Exemplary techniques are provided below and in the Examples section.

### Determination of Anti-Inflammatory Activity In Vitro

As is known in the art, keratinocytes are responsible for the immune surveillance from exogenous or intrinsic stress. Accordingly, the effect of the Tsuga extracts on the release of cytokines such as Interleukin-1 alpha (IL-1) as part of the inflammatory response can be assayed. This assay employs a model comprising keratinocytes in a growth medium that includes a suitable amount of the Tsuga extract. After an appropriate incubation period, the keratinocytes are exposed to UV light to induce an inflammatory response and are subsequently incubated for a further period of time, for example, about 24 hours. The amount of IL-1 produced by the keratinocytes in response to the UV treatment can be assessed, for example, by a specific ELISA that quantitates IL-1. The amount of IL-1 can be compared to an untreated control and/or to a control treated with a compound known to inhibit inflammation.

The anti-inflammatory effects of the Tsuga extracts can also be tested in an in vitro psoriasis model. As is known in the art, keratinocytes undergo differentiation in the different layers of the epidermis. In the process, several markers are expressed that reflected the level of differentiation. Accordingly, the effect of the Tsuga extracts on the expression of such markers on psoriatic-like induced keratinocytes can be assayed. This assay employs a model comprising keratinocytes in a rich serum growth media that includes a suitable amount of the Tsuga extract. After an appropriate incubation period, the keratinocytes are fixed with formaldehyde and assayed for different differentiation markers such as involucrin, Transglutaminase, cytokeratin-10 and 16 and/or SKALP, for example, using indirect immunofluorescence. The amount of each marker can be evaluated qualitatively or quantitatively. The level of markers induced in keratinocytes treated with the Tsuga extract can be compared to an untreated control and/or to a control treated with a compound known to treat psoriasis, such as dithranol.

### Determination of Anti-Inflammatory Activity In Vivo

The ability of the plant extracts to reduce inflammation in the skin can be assessed in human volunteers using standard techniques. For example, the Minimal Erythema Dose (MED) test measures the minimum UV dose required to produce a distinct reaction in the skin in the form of redness, which appears 24 hours after exposure. The MED of all volunteers must be measured prior to any treatment that attempts to measure the skin response to UV induced erythema.

In general, at Day D0, the skin color for each volunteer is measured in the test zone (usually the forearm) using a Mexameter® to approximate the MED of the individual volunteer. Actinic erythema is induced using a UV simulator to the selected zone by applying a series of 5 consecutive increasing doses of irradiation to two sites on the skin—the first, a non-treated site on one forearm and the second, a site treated with the Tsuga extract (typically formulated into a cream) on the other forearm. The skin response on each forearm is then assessed visually 24 hrs later (Day D1) to calculate the individual MED (the lowest dose to which a distinct reaction is observed).

Other tests, such as the sodium lauryl sulphate (SLS) patch test and the Mantoux test, which involves the injection of tuberculin, are known in the art and may be employed to test the anti-inflammatory effects of the Tsuga extracts. Various noninvasive measuring methods can be used to evaluate the skin before and after exposure to the applied irritants, for example, measurement of transepidermal water loss by an evaporimeter, measurement of electrical conductance by a hydrometer, measurement of skin blood flow by laser Doppler flowmetry, measurement of skin colour using a colorimeter and/or measurement of skin thickness by ultrasound A-scan (see, for example, Agner, T., (1992) Acta Derm Venereol Suppl (Stockh). 173:1-26, and De Fine Olivarious, F., et al. (1993) Br J Dermatl. 129:554-557).

Attenuation of psoriasis lesions can be evaluated by conducting tests using a panel of human volunteers with psoriatic plaques. The test typically involves application of the Tsuga extract (typically formulated into a cream) to the affected areas of the patients' skin on a regular basis, such as once or twice a day, over a period of several weeks. The effect of the Tsuga extract on the psoriasis plaques can be evaluated by visually inspecting the treated plaques and assessing the characteristic or characteristics being investigated, for example, decrease of redness, lesions and/or desquamation. Other aspects, such as decrease in itchiness can also be investigated.

### Determination of Anti-Microbial Activity In Vitro

The ability of the Tsuga extracts to inhibit the growth of microbial cells can be assessed using standard in vitro methods known in the art. In general, these methods involve contacting a culture of the microbial cells with various concentrations of the extract and monitoring the growth of the cell culture relative to an untreated control culture. A second control culture comprising cells contacted with a known anti-microbial agent may also be included in such tests, if desired. An exemplary test for determining the ability of the Tsuga extract to inhibit a variety of bacterial species is provided in the Examples (see Example XI).

The ability of the Tsuga extracts to inhibit the growth of bacterial cells can also be determined by measurement of the minimum inhibitory concentration (MIC). The MIC is defined as the lowest concentration that inhibits growth of the organism to a pre-determined extent. For example, a MIC₁₀₀ value is defined as the lowest concentration that completely inhibits growth of the organism, whereas a MIC₉₀ value is defined as the lowest concentration that inhibits growth by 90% and a MIC₅₀ value is defined as the lowest concentration that inhibits growth by 50%. MIC values are sometimes expressed as ranges, for example, the MIC₁₀₀ for a compound may be expressed as the concentration at which no growth is observed or as a range between the concentration at which no growth is observed and the concentration of the dilution which immediately follows.

Typically, anti-bacterial MICs are measured using a broth macro- or microdilution assay (see Amsterdam, D. (1996) “Susceptibility testing of antimicrobials in liquid media,” pp. 52-111. In Loman, V., ed. Antibiotics in Laboratory Medicine, 4th ed. Williams and Wilkins, Baltimore, Md.). A standardised anti-bacterial susceptibility test is provided by the National Committee for Clinical Laboratory Standards (NCCLS) as NCCLS, 2000; document M7-A58.

In the classical broth microdilution method, the Tsuga extract would be diluted in culture medium in a sterile, covered 96-well microtiter plate. An overnight culture of a single bacterial colony is diluted in sterile medium such that, after inoculation, each well in the microtiter plate contains an appropriate number of colony forming units (CFU)/ml (typically, approximately 5×10⁵ CFU/ml). Culture medium only (containing no bacteria) is also included as a negative control for each plate and known antibiotics are often included as positive controls. The inoculated microtiter plate is subsequently incubated at an appropriate temperature (for example, 35° C.-37° C. for 16-48 hours). The turbidity of each well is then determined by visual inspection and/or by measuring the absorbance, or optical density (OD), at 595 nm or 600 nm using a microplate reader and is used as an indication of the extent of bacterial growth.

### In Vitro Cellular Activity

The ability of the Tsuga extracts to affect one or more of angiogenesis, contractile force of fibroblasts or UV-induced protease activity can be assessed in vitro using one, or a combination, of standard techniques known in the art.

For example, inhibition of angiogenesis can be assessed using the cord formation assay. In this assay, endothelial cells with or without the Tsuga extract are plated onto Matrigel® and incubated under appropriate conditions. After a suitable period of time, (for example, between 18 and 24 hours), the evaluation of angiogenesis is assessed by visual inspection to determine whether the cells have formed into cords.

Various cell lines can be used in cord formation assays. Examples of suitable endothelial cell lines include, but are not limited to, human umbilical vein endothelial cells (HUVECs), bovine aortic endothelial cells (BAECs), human coronary artery endothelial cells (HCAECs), bovine adrenal gland capillary endothelial cells (BCE) and vascular smooth muscle cells. HUVECs can be isolated from umbilical cords using standard methods (see, for example, Jaffe et al. (1973) J. Clin. Invest. 52(11): 2745-56), or they can be obtained from the ATCC or various commercial sources, as can other suitable endothelial cell lines.

The effect of the Tsuga extracts on the tractional forces generated by fibroblasts can be assayed, for example, using a model comprising fibroblasts embedded in a collagen matrix to create a derm-like environment. Such a model can be prepared by adding fibroblasts to a solution of collagen I in medium and then allowing the collagen to polymerize to form a gel. After an appropriate incubation period, the derm-like gel is treated with the Tsuga extract and the amount of contraction measured over a period of time, for example, several days. The amount of contraction can be assessed for example, by digitally photographing the gel at various time points and calculating the gel area using appropriate software. The amount of contraction can be compared to untreated control gels and/or gels treated with a compound known to affect fibroblast tractional forces.

UV-induced extracellular protease activity can be assessed by irradiating cultures of skin cells with UVA light and then treating the irradiated cells with the Tsuga extract. Alternatively, the extract can be added to the cells prior to irradiation to assess the prophylactic effect of the extract. After a suitable period of incubation in an appropriate medium, supernatants can be removed from the cells and assayed for proteolytic activity, such as MMP-9 activity as described below. Results can be compared to untreated cells and/or cells treated with a compound known to affect UV-induced protease activity.

Skin cells suitable for use in the above assay include human dermal fibroblasts, keratinocytes, melanocytes, Langerhans cells, cells of the hair follicle and cells of the immune system which produce proteases, including leukocytes, macrophages and lymphocytes.

### In Vivo Cellular Activity

The Tsuga extracts may undergo additional testing on human volunteers to assess their ability to exert the desired dermatological effect(s).

For example, the effect of the extracts on skin changes, such as wrinkling and/or sagging, reddening, formation of lesions, abnormal pigmentation and the like, can be assessed by visual examination. For example, the effect of the Tsuga extract on the skin can be evaluated by formulating the extract such that it is suitable for external application to the skin and subsequently sensory tests can be conducted on the formulation using a panel of human volunteers. A sensory test typically involves application of the formulation to the skin of the panelists on a regular basis, such as once or twice a day, over a period of several weeks. The effect of the formulation on the skin can be evaluated by inspecting the skin of the panelists and assessing visually the skin characteristic or characteristics being investigated, for example, the tenseness and gloss of the skin, the appearance of existing wrinkles and sags, reddening, lesions and/or abnormal pigmentation. Attenuation of Crow's feet can be determined, for example, using a computerized digital image to obtain a skin's topography before and after treatment in human volunteers. Erythema in skin samples can be determined, for example, using commercially available chromameter.

The plant extracts may also undergo one or more safety, stability and/or bioavailability test prior to testing on human volunteers, for example, the Human Repeat Insult Patch Test (HRIPT).

### Other Tests

### Determination of MMP-9 Inhibitory Activity

A variety of methods and techniques for measuring the ability of the Tsuga extracts to inhibit the activity of MMP-9 either qualitatively or quantitatively are known in the art. For example, there are currently several assays to measure the activity of MMPs (including MMP-9) (for a review of these methods, see Murphy and Crabbe, In Barrett (ed.) Methods in Enzymology. Proteolytic Enzymes: Aspartic Acid and Metallopeptidases, New York: Academic Press, 1995, 248: 470), including the gelatineolytic assay (which is based on the degradation of radio-labelled type I collagen), the zymography assay (which is based on the presence of negatively-stained bands following electrophoresis through substrate-impregnated SDS polyacrylamide gels) and a microtitre plate assay developed by Pacmen et al., (Biochem. Pharm. (1996) 52:105-111).

Other methods include those that employ auto-quenched fluorogenic substrates. Many fluorogenic substrates have been designed for quantification of the activity of MMPs through fluorescent level variation measuring (reviewed by Nagase and Fields (1996) Biopolymers 40: 399-416). Another method of measuring MMP-9 activity makes use of the fluorescent activated substrate conversion (FASC) assay described in Canadian Patent No. 2,189,486 and in St-Pierre et al. ((1996) Cytometry 25: 374-380).

Alternatively, the ability of the Tsuga extracts to inhibit the activity of MMP-9 can be evaluated using cultures of cells that secrete MMPs. In this case a cell culture is in contact with an appropriate amount of the extract. After an appropriate period of time, the cells are extracted, centrifuged and the proteolytic activity in the supernatant is measured. This method is useful in determining the ability of an extract to inhibit MMP-9 secreted by a particular cell line or combination of cell lines. For example, assays can be conducted with cell lines derived from mammalian skin, such as keratinocytes or fibroblasts.

### In Vivo Testing of MMP-9 Inhibition

The ability of the Tsuga extracts to inhibit MMP-9 activity in vivo may be assessed using various standard techniques. For example, the ability of the Tsuga extracts to inhibit protease activity can be determined in animal models or human volunteers. An example of a suitable animal model would be a skh-1 mouse or nude mouse or rat that is treated with a Tsuga extract and then exposed to UV radiation (see, Nishimori et al. (2001) J. Invest. Dermatol. 117:1458-1463). UV radiation is known to increase the level of activity of certain MMPs (see, for example, U.S. Pat. No. 6,130,254). Skin biopsies are taken from the animal and the amount of MMP-9 activity in the biopsied sample can be measured using standard techniques as an indication of the inhibitory activity of the test extract.

Human trials may also be used to evaluate the ability of the Tsuga extract to inhibit MMP-9 activity in the skin. For example, skin biopsies can be taken from adult volunteers exposed to UV radiation and treated prior to or after UV exposure with an extract. The biopsy samples can be assessed for MMP-9 activity and compared to an appropriate control (for example, skin biopsies from individuals treated with a control compound or untreated individuals). Alternatively, as an age-related increase in the relative activities of MMP-1, MMP-2 and MMP-9 has been demonstrated (see, for example, U.S. Patent Application No. 20010053347), elderly individuals (for example, those over 80 years of age) could be used as volunteers for the trials without the requirement for UV exposure.

In order to assess the protease activity in skin biopsies, the samples are typically flash frozen, mechanically ground and/or homogenised. After centrifugation, the supernatants are isolated and used to assess MMP-9 activity in assays such as those outlined above.

### Safety Testing

In addition to the above tests, the Tsuga extracts may be submitted to other standard tests to evaluate safety, cytotoxicity, stability, bioavailability and the like. Exemplary tests to determine the cytotoxicity of the extracts and their potential to induce cytokine release are described herein (see Examples III and VI).

The ability of the Tsuga extract to penetrate the skin can be assessed, for example, by in vitro release tests (see, for example, the U.S. Center for Drug Evaluation and Research guidance document entitled “Guidance for Industry, Nonsterile Semisolid Dosage Forms. Scale-up and postapproval changes: in vitro release testing and in vivo binequivalence documentation”). Typically, such testing is conducted using an open chamber diffusion cell, such as a Franz cell, fitted with an appropriate membrane. The test extract is placed on the upper side of the membrane and kept occluded to prevent solvent evaporation and compositional changes. A receptor fluid, such as aqueous buffer or hydro-alcoholic medium, is placed on the other side of the membrane in a receptor cell. Diffusion of the active component across the membrane is monitored by assay of sequentially collected samples of the receptor fluid. For the Tsuga extracts in accordance with the invention, the assay could comprise, for example, testing the ability of the collected sample to inhibit MMP-9 activity. The membrane can be a synthetic membrane, for example polysulphone, cellulose acetate or nitrate, or polytetrafluoroethylene, or it can be a skin sample, such as a sample taken from a cadaver.

Other tests are known in the art (for example, see U.S. Pharmacopoeia XXII (1990)) and are suitable for testing the stability and/or safety of the Tsuga extracts.

As will be readily apparent to one skilled in the art, a selected extract may need to meet certain criteria in order to meet regulatory requirements for human use. Conducting tests such as those described above, therefore, allows the suitability of an extract for human use to be assessed.

### Dermatological Formulations

The present invention further provides for formulations comprising a Tsuga extract suitable for dermatological, including cosmetic, applications. The formulations can optionally comprise other active agents, such as therapeutic or cosmetic agents and/or other plant extracts. The formulations are prepared by standard techniques such that they have acceptable toxicity and stability.

The formulations are prepared by mixing a suitable amount of the Tsuga extract with a physiologically acceptable carrier or diluent. In one embodiment of the invention, the formulation comprises only the Tsuga extract and a diluent or carrier. In another embodiment of the invention, the formulation comprises the Tsuga extract and one or more thickener, excipient, binder or the like, and optionally one or more other active agent.

Suitable amounts of the Tsuga extract for incorporation into the dermatological formulation are generally in the range of between about 0.1% (v/v) and about 20% (v/v). In one embodiment, the dermatological formulation comprises between about 0.1% (v/v) and about 18% (v/v) of the Tsuga extract. In another embodiment, the dermatological formulation comprises between about 0.1% (v/v) and about 16% (v/v) of the Tsuga extract. In another embodiment, the dermatological formulation comprises between about 0.1% (v/v) and about 15% (v/v) of the Tsuga extract. In other embodiments, the dermatological formulation comprises between about 0.1% (v/v) and about 14% (v/v), between about 0.1% (v/v) and about 12% (v/v), between about 0.1% (v/v) and about 10% (v/v), between about 0.1% (v/v) and about 8% (v/v), and between about 0.1% (v/v) and about 6% (v/v) of the Tsuga extract.

In other embodiments, the dermatological formulation comprises between about 0.2% (v/v) and about 20% (v/v), between about 0.4% (v/v) and about 20% (v/v), between about 0.5% (v/v) and about 20% (v/v), between about 0.5% (v/v) and about 18% (v/v), between about 0.6% (v/v) and about 15% (v/v), between about 0.8% (v/v) and about 12% (v/v), and between about 1.0% (v/v) and about 10% (v/v) of the Tsuga extract.

The amount of Tsuga extract for incorporation into the dermatological formulation can also be defined as % by weight. For example, suitable amounts of the Tsuga extract for incorporation into the dermatological formulation are generally in the range of between about 0.1% and about 20% by weight. In various embodiments of the invention, the dermatological formulation comprises between about 0.1% and about 18% by weight, between about 0.1% and about 16% by weight, between about 0.1% and about 15% by weight, between about 0.1% and about 14% by weight, between about 0.1% and about 12% by weight, between about 0.1% and about 10% by weight, between about 0.1% and about 8% by weight, between about 0.1% and about 6% by weight, between about 0.2% and about 20% by weight, between about 0.4% and about 20% by weight, between about 0.5% and about 20% by weight, between about 0.5% and about 18% by weight, between about 0.6% and about 15% by weight, between about 0.8% and about 12% by weight, and between about 1.0% and about 10% by weight of the Tsuga extract.

In one embodiment of the present invention, the dermatological formulations comprising the Tsuga extract are formulated for topical administration. Such formulations may be presented as, for example, aerosol sprays, powders, sticks, granules, creams, liquid creams, mousses, pastes, gels, lotions, ointments, on sponges or cotton applicators, or as a solution or a suspension in an aqueous liquid, a non-aqueous liquid, an oil-in-water emulsion, or a water-in-oil liquid emulsion.

Various physiologically acceptable carriers are known in the art. Examples of such carriers include, but are not limited to, hydroxypropyl cellulose, starch (corn, potato, rice, wheat, quinoa), pregelatinized starch, gelatine, sucrose, acacia, alginic acid, sodium alginate, guar gum, ethyl cellulose, carboxymethylcellulose sodium, carboxymethylcellulose calcium, polyvinylpyrrolidone, methylcellulose, hydroxypropyl methylcellulose, microcrystalline cellulose, polyethylene glycol, powdered cellulose, glucose, croscarmellose sodium, crospovidone, polacrilin potassium, sodium starch glycolate, tragacanth, calcium carbonate, dibasic calcium phosphate, tribasic calcium phosphate, kaolin, mannitol, talc, cellulose acetate phthalate, polyethylene phthalate, shellac, titanium dioxide, carnauba wax, microcrystalline wax, calcium stearate, magnesium stearate, castor oil, mineral oil, light mineral oil, glycerine, sorbitol, mannitol, stearic acid, sodium lauryl sulfate, hydrogenated vegetable oil (for example, peanut, cottonseed, sunflower, sesame, olive, corn, soybean), zinc stearate, ethyl oleate, ethyl laurate, agar, calcium silicate, magnesium silicate, silicon dioxide, colloidal silicon dioxide, calcium chloride, calcium sulfate, silica gel, castor oil, diethyl phthalate, glyercine, mono- and di-acetylated monoglycerides, propylene glycol, triacetin, alamic acid, aluminum monostearate, bentonite, bentonite magma, carbomer 934, carboxymethylcellulose sodium 12, carrageenan, hydroxyethyl cellulose, magnesium aluminum silicate, pectin, polyvinyl alcohol, povidine, sodium alginate, tragacanth, xanthan gum, silicones and various combinations thereof.

Exemplary thickeners are cross-linked polyacrylate materials available under the trademark Carbopol™ (B. F. Goodrich Company), xanthan gum, carrageenan, gelatine, karaya, pectin and locust bean gum. Under certain circumstances the thickening function may be accomplished by a moisturiser component of the formulation. For instance, silicone gums and esters such as glycerol stearate have dual functionality. A thickener will usually be present in amounts from 0.1 to 20% by weight of the formulation.

The formulations can optionally include one or more moisturising agents, i.e. an agent that facilitates hydration of the skin by inhibiting or preventing loss of water from the skin, that absorbs water from the atmosphere and hydrates the skin, and/or that enhances the skin's ability to absorb water directly from the atmosphere. Moisturising agents generally minimise or prevent the skin from drying and cracking. Moisturisers, when used, are typically present in an amount from about 0.01 to 20% by weight of the formulation.

Examples of moisturising agents include, but are not limited to, 2-hydroxyacetic acid (glycolic acid); 2-hydroxypropanoic acid (lactic acid); 2-methyl 2-hydroxypropanoic acid; 2-hydroxybutanoic acid; phenyl 2-hydroxyacetic acid; phenyl 2-methyl 2-hydroxyacetic acid: 3-phenyl 2-hydroxyacetic acid; 2,3-dihydroxypropanoic acid; 2,3,4-trihydroxybutanoic acid; 2,3,4,5,6-pentahydroxyhexanoic acid; 2-hydroxydodecanoic acid; 2,3,4,5-tetrahydroxypentanoic acid; 2,3,4,5,6,7-hexahydroxyheptanoic acid; diphenyl 2-hydroxyacetic acid; 4-hydroxymandelic acid; 4-chloromandelic acid; 3-hydroxybutanoic acid; 4-hydroxybutanoic acid; 2-hydroxyhexanoic acid; 5-hydroxydodecanoic acid; 12-hydroxydodecanoic acid; 10-hydroxydecanoic acid; 16-hydroxyhexadecanoic acid; 2-hydroxy-3-methylbutanoic acid; 2-hydroxy-4-methylpentanoic acid; 3-hydroxy-4-methoxymandelic acid; 4-hydroxy-3-methoxymandelic acid; 2-hydroxy-2-methylbutanoic acid; 3-(2-hydroxyphenyl) lactic acid; 3-(4-hydroxyphenyl) lactic acid; hexahydromandelic acid; 3-hydroxy-3-methylpentanoic acid; 4-hydroxydecanoic acid; 5-hydroxydecanoic acid; aleuritic acid; 2-hydroxypropanedioic acid; 2-hydroxybutanedioic acid; tannic acid; salicylic acid; erythraric acid; threaric acid; arabiraric acid; ribaric acid; xylaric acid; lyxaric acid; glucaric acid; galactaric acid; mannaric acid; gularic acid; allaric acid; altraric acid; idaric acid; talaric acid; 2-hydroxy-2-methylbutanedioic acid; citric acid, isocitric acid, agaricic acid, quinic acid, glucoronic acid, glucoronolactone, galactoronic acid, galactoronolactone, uronic acids, uronolactones, ascorbic acid, dihydroascorbic acid, dihydroxytartaric acid, tropic acid, ribonolactone, gluconolactone, galactonolactone, gulonolactone, mannonolactone, citramalic acid; pyruvic acid, hydroxypyruvic acid, hydroxypyruvic acid phosphate and esters thereof; methyl pyruvate, ethyl pyruvate, propyl pyruvate, isopropyl pyruvate; phenyl pyruvic acid and esters thereof; methyl phenyl pyruvate, ethyl phenyl pyruvate, propyl phenyl pyruvate; formyl formic acid and esters thereof; methyl formyl formate, ethyl formyl formate, propyl formyl formate; benzoyl formic acid and esters thereof; methyl benzoyl formate; ethyl benzoyl formate; propyl benzoyl formate; 4-hydroxybenzoyl formic acid and esters thereof; 4-hydroxyphenyl pyruvic acid and esters thereof; and 2-hydroxyphenyl pyruvic acid and esters thereof. It should be understood that one or more derivatives of the above compounds, including esters or lactones or pharmaceutically acceptable salts thereof, may also be used.

Further examples of moisturising agents include, but are not limited to beeswax, shea butter, ceramide, borage oil (linoleic acid), tocopherol linoleate, dimethicone, glycerine, hyaluronic acid, sodium peroxylinecarbolic acid (sodium PCA), wheat protein (such as laurdimonium hydroxypropyl hydrolyzed wheat protein), primrose oil, flax seed oil and mixtures thereof.

As noted above, the dermatological compositions may optionally include one or more other active agents and/or plant extracts that are intended to impart additional beneficial properties to the formulation. For example, the dermatological formulation can also include one or more additional anti-inflammatory components which facilitate inhibition or suppression of inflammation on or in the skin or in adjacent bodily tissues and thereby help to reduce redness and swelling of the skin. Examples of suitable anti-inflammatory components include vitamin E and derivatives thereof, zinc, allantoin, glycyrrhetic acid, azulene, mefenamic acid, phenylbutazone, indometacin, ibuprofen, ketoprofen, aminocaproic acid, hydrocortisone, panthenol and derivatives and salts thereof, zinc oxide, salicylic acid and diclofenac sodium. The anti-inflammatory component, when used, can be incorporated into the formulations of the present invention in an amount between about 0.001 to about 5% by weight.

Other examples of skin benefit ingredients that may be included in the formulations in various embodiments of the invention include, but are not limited to, anti-oxidants, retinols and retinol derivatives (including pro-retinol), alpha-hydroxy acids (AHAs), skin whitening agents and other anti-ageing ingredients. Examples of retinol and retinol derivatives include, but are not limited to, retinol, retinoic acid (tretinoin), isotretinoin and retinaldehyde. Examples of AHAs include, but are not limited to, lactic acid and glycolic acid. Examples of skin whitening agents include, but are not limited to kojic acid, kojic dipalmitate, hydroquinone, arbutins (such as alpha-arbutin, beta-arbutin and deoxy-arbutin) and azelaic acid. Tretenoin is also known to have slight skin whitening properties and may be used in combination with another skin whitening agent.

The formulations may also optionally comprise one or more of a sunscreen, a sunblock, essential fatty acids, a cell activator, a blood-circulation promoter, a tanning agent, proteins, peptides and polysaccharides.

Azoles, such as climbazole, bifonazole, clotrimazole, ketoconazole, miconazole, econazole, itraconazole, fluconazole, terconazole, butoconazole, sulconazole, lionazole and mixtures thereof, may also optionally be included in the formulations.

In one embodiment of the invention, the dermatological formulation comprises one or more additional anti-ageing ingredients. In another embodiment, the dermatological formulation comprises retinol or a retinol derivative. In another embodiment, the dermatological formulation comprises an AHA. In another embodiment, the dermatological formulation comprises retinol or a retinol derivative or an AHA in a higher than standard amount. As is known in the art, retinol is generally added to skin care formulations in amounts between about 0.1% and about 0.5% by weight, and AHAs are generally added in amounts between about 1% and about 6% by weight. Kojic acid or hydroqxuinone are generally included in dermatological compositions in amounts up to 4% by weight. Accordingly, in the context of the present invention a “higher than standard amount” of retinol is considered to be 0.5% by weight or greater, and a “higher than standard amount” of an AHA is considered to be 5% by weight or greater.

In one embodiment, the formulation comprises 0.6% by weight or higher of retinol or a retinol derivative. In other embodiments, the formulation comprises 0.7% by weight or higher, 0.8% by weight or higher, 0.9% by weight or higher, or 1.0% by weight or higher, of retinol or a retinol derivative. In one embodiment, the formulation comprises 6% by weight or higher of an AHA. In other embodiments, the formulation comprises 6.5% by weight or higher, 7.0% by weight or higher, 7.2% by weight or higher, 7.5% by weight or higher, 7.8% by weight or higher, or 8.0% by weight or higher, of an AHA. In one embodiment, the formulation comprises 4% by weight or higher of a whitening agent, such as kojic acid, kojic dipalmitate or hydroquinone. In other embodiments, the formulation comprises 4.5% by weight or higher, 5.0% by weight or higher, 5.2% by weight or higher, 5.5% by weight or higher, 6.0% by weight or higher of a whitening agent, such as kojic acid, kojic dipalmitate or hydroquinone.

In another embodiment, the dermatological formulation comprises one or more other plant extracts.

Other adjunct minor components can also optionally be incorporated into the dermatological formulations, for example, colouring agents, opacifiers, perfumes and preservatives (for example, imidazolidinyl urea, dimethyl imidazolidinone or diazolidinyl urea). Amounts of these materials can range from 0.001% up to 20% by weight of the formulation.

The dermatological formulations intended for topical application can be packaged in a suitable container to suit the viscosity and intended use. For example, a lotion, fluid cream, foam or mousse can be packaged in a bottle or a roll-ball applicator, a capsule, a propellant-driven aerosol device or a container fitted with a pump suitable for finger operation. When the composition is a cream or paste, it can simply be stored in a non-deformable bottle or squeeze container, such as a tube or a lidded jar.

### Use

The present invention provides for the use of the Tsuga extracts and formulations comprising same to ameliorate inflammation, irritation and/or infection in a subject. In one embodiment, the invention provides for the use of to ameliorate the effects of inflammation, irritation or infection, for example, by promoting the formation of scar tissue and/or promoting healing of wounds caused by the inflammation, irritation or infection.

For example, the Tsuga extracts of the invention and dermatological formulations comprising the Tsuga extracts are suitable for a variety of applications in the dermatological field. For example, in one embodiment, the invention provides for the use of the Tsuga extracts and formulations for the treatment of dermatological conditions and, in particular, dermatological conditions with an associated inflammatory component or infection. For example, the extracts and formulations can be used to treat psoriasis, rosacea, acne, dermatitis (for example, atopic dermatitis, professional dermatitis and/or seborrheic dermatitis), erythema, eczema, pruritis, lichen simplex, photodermatosis (for example, polymorphous light eruption and actinic prurigo), prurigo nodulans, sunburn, hives, and rashes (including diaper rash). The extracts and formulations can also be used for treating obesity-related skin problems (for example, inflammation, redness, erythema, rashes and/or bacterial infections caused by skin folds) and for combating the irritant or inflammatory effects of, or infection due to, cosmetic or surgical skin procedures, such as peeling, exfoliation, laser treatments, hair removal or plastic surgery.

In one embodiment, the invention provides for the use of the Tsuga extracts and formulations comprising same for the treatment of acne. In another embodiment, the invention provides for the use of the Tsuga extracts and formulations comprising same for the treatment of erythema. Erythema can be caused by, for example, infection, massage, electrical treatment, acne medication, allergies, exercise, solar radiation (such as sunburn), cutaneous radiation syndrome, hair removal (for example, plucking, waxing, laser removal), laser treatments or radiotherapy.

In another embodiment, the invention provides for the use of the Tsuga extracts in formulations comprising skin treatment compounds that can exert irritant or inflammatory effects on the skin of the user, for example, retinol, retinol derivatives, AHAs and/or skin whitening agents, in order to ameliorate the irritant/inflammatory effects of these compounds. In another embodiment, the invention provides for the use of the Tsuga extracts in dermatological formulations for sensitive skins. In another embodiment, the invention provides for the use of the Tsuga extracts in dermatological formulations for ameliorating the irritant/inflammatory effects of environmental factors, for example in “after sun” formulations, and/or of cosmetic skin procedures, for example in “after shave” formulations.

The Tsuga extracts can also be incorporated into dermatological formulations in order to provide a preservative effect due to their anti-microbial activity.

One embodiment of the present invention provides for the use of the Tsuga extract as an anti-inflammatory agent. Another embodiment of the invention provides for the use of the Tsuga extract as an anti-microbial agent. Another embodiment of the invention provides for the use of the Tsuga extract to ameliorate the inflammatory and/or irritation symptoms of a dermatological condition.

Another embodiment of the present invention provides for the use of the Tsuga extract in an anti-ageing product. An anti-ageing product is a product intended for use to attenuate skin ageing that occurs due to intrinsic or extrinsic factors. The anti-ageing product may comprise other anti-ageing compounds in addition to the Tsuga extract. Skin ageing phenomena that can be treated with anti-ageing products include, for example, skin thinning, fine and coarse skin wrinkling, sagging, loss of elasticity, and the like.

Another embodiment of the invention provides for the use of the Tsuga extract and formulations comprising the Tsuga extract to promote the formation of scar tissue. A further embodiment provides for the use of the Tsuga extract and formulations comprising the Tsuga extract to promote wound healing.

To gain a better understanding of the invention described herein, the following examples are set forth. It should be understood that these examples are for illustrative purposes only. Therefore, they should not limit the scope of this invention in any way.

### EXAMPLES

### Example I

### Preparation of Extracts from Tsuga canadensis

### Analytical Scale Preparation

In general, five grams of the dried plant material to be extracted was placed in a beaker and a sufficient amount of solvent was added to allow moderate agitation with a stirring bar. The solvents used in this Example were: butylene glycol (100%), butylene glycol/water (50/50, v/v), butylene glycol/water (20/80, v/v); ethanol (100%), ethanol/water (85/15, v/v), ethanol/water (50/50, v/v); water (100%); 1,3-propanediol (100%); 1,3-propanediol/water (50/50, v/v) and 1,3-propanediol/water (20/80, v/v).

Several different extraction times were employed for each solvent: after mixing for periods of 4 to 24 hours at room temperature, the suspension was filtered through a 60 mesh filter and then filtered through 40, 20, 11 and 1.2 micron paper filter consecutively. For the filtered butylene glycol mixtures, the solvent was then evaporated at 120° C. and the residual matter was weighed to determine the yield of extraction at each time point. For the filtered ethanol mixtures, the solvent was removed under reduced pressure at a temperature of less than 45° C. in order to determine the yield of extraction at each time point.

The above protocol is suitable for the preparation of extracts that are to be employed in dermatological formulations. Glycol extracts such as butylenes glycol or 1,3-propanediol extracts, for example, can be included directly into formulations intended for topical application. Ethanol extracts may undergo one or more additional steps, such as CO₂ extraction, prior to incorporation into formulations intended for topical application.

The following is an example of a specific protocol that was employed to prepare a Tsuga canadensis extract.

To 5 g of dried needles and small branches of Tsuga canadensis lot 20252, 100 ml of a 50:50 butylene glycol:water was added. The mixture was thoroughly stirred and macerated for 4 hours at room temperature. The resulting mix was filtered through 40μ to 1.2μ filters to obtain a suitable extract for further analysis.

The Tsuga canadensis extracts 20252-PLA; 20156A; 207-20156A and 20010 1NG13B tested in the following examples are all extracts prepared from needles and small branches of Tsuga canadensis using 50:50 butylene glycol:water as the extraction solvent following the protocol outlined above. The extracts differed in the individual Tsuga canadensis tree that the plant material came from and in the time that the plant material was harvested.

The Tsuga canadensis extract 20376 1NG23ZB also tested in the following examples are extracts prepared from needles of Tsuga canadensis using 50:50 1.3-propanediol:water as the extraction solvent following the protocol outlined above.

### Large Scale Preparation

Analysis of the results from the analytical scale preparation allows for the selection of appropriate plant materials for the large-scale extraction. The selection includes a decision regarding part of the Tsuga tree and quantity of dried material needed to obtain a sufficient yield on a large scale. The selection also involves a choice of solvent system for an active extract.

The extraction protocol is essentially the same as the procedure for the analytical preparation except for the filtration process. The dried and pulverized material (2-3 Kg for large scale) is extracted repeatedly (maceration/percolation) with solvent (3×2-20 l) at room temperature for 24-72 h, based on the analytical scale yield of extraction results. The resulting extract is then passed through a 60 mesh filter and then may be filtered through 10 to 100 kDa ultrafiltration column or on a 0.1 to 1μ ceramic column.

Non-limiting examples of large scale extraction preparations of extracts from Tsuga canadensis are provided below.

A. To 3 kg of dried needles and small branches from Tsuga canadensis lot 20252, 60 L of a 50:50 Butylene glycol:water was added. The mixture was thoroughly stirred and macerated for 24 hours. The resulting mix was filtered through a 65 mesh filter and then filtered on a 1μ ceramic column to obtain an extract suitable for further analysis.

B. To 3 kg of dried needles and small branches from Tsuga canadensis lot 20252, 60 L of a 50:50 Butylene glycol:water or 1,3-propanediol:water was added. The mixture was thoroughly stirred and macerated for 24 hours, then decanted for another 18-24 hours. The resulting mix was filtered through a 65 mesh filter and then filtered by ultrafiltration at 100 kDa to obtain an extract suitable for further analysis.

### Example II

### In Vitro MMP-9 Inhibition Assays

MMP-9 was purified from natural sources (human cell lines, THP-1 (ATCC, Manassas, Va.)) as described in literature and based on protocols found in I. M. Clark: “Matrix metalloproteinases protocols”, Humana Press (2001). Proteolytic activity of the protease was evaluated with the assay based on the cleavage of auto-quenched peptide substrate: (MCA-Pro-Leu-Gly-Leu-Dpa-Ala-Arg-NH₂.TFA [Dpa=N-3-(2,4-dinitrophenyl)-L-2,3-diaminopropionyl]). In the intact peptide, Dpa or DNP quenches the MCA fluorescence. Cleavage of the peptide causes release of the fluorescent MCA group which was then quantified on a fluorometer (Gemini XS, Molecular Devices, Sunnyvale, Calif.). The assay was performed in TNCZ assay buffer (20 mM Tris-HCl; NaCl 150 mM: CaCl₂ 5 mM: ZnCl₂ 0.5 mM: pH 7.5) with purified human MMP-9 (I. M. Clark: Matrix metalloproteinases protocols, Humana Press (2001)). The substrate, primarily dissolved in DMSO was then redissolved in TNCZ buffer for the assay.

In a typical fluorescent assay, 10 μl of purified enzyme at concentrations previously mentioned for the enzymatic assay, 5 μl of dissolved fluorogenic peptide and 40 μL of the Tsuga extract to be tested were mixed in a final volume of 75 μl, completed with TNCZ. All assays were performed in 96 well plates and the reaction was started by the addition of substrate. Assays were measured (excitation 325 nm, emission 392 nm) for 20, 40 and 60 minutes. Activity and inhibition values were determined from the increase in fluorescence.

The inhibition is reported as percentage (%) of inhibition of substrate degradation as compared with substrate degradation in the absence of the Tsuga extract. Percentage inhibition was calculated according to the formula:

Percentage (%) inhibition=[E_{A}−E_{B}/E_{A}]×100

wherein E_{A} is the protease activity in the absence of the plant extract and E_{B} is the protease activity in the presence of the extract.

Following the above protocol, Tsuga canadensis extract 20156A (a 50:50 Butylene glycol:water extract) was shown to inhibit MMP-9 activity by 50% at a concentration of 25 μg/ml, and Tsuga canadensis extract 20252-PLA inhibited MMP-9 activity by 50% at 156 μg/ml.

### Example III

### Viability/Growth Assay

This example provides results reflecting the non-cytotoxic concentration of Tsuga extracts that can be used for further in vitro efficacy studies.

### Growth Evaluation of Human Skin Cells in the Presence of Tsuga Extracts

Normal human skin fibroblasts and keratinocytes (Cascade Biologics, Portland, Oreg.) were tested to evaluate the anti-proliferative effect of the Tsuga extracts. The latter was done to ascertain that the exposure of cells to a concentration of extract would have no undesirable effect for further cellular assays.

Cells (5×10³ cells/100 μl/well for fibroblasts and 8×10³ cells/100 μl/well for keratinocytes) were seeded in a 96-well plate in their respective media and then incubated for 24 hours at 37° C. in a humidified 5% CO₂ atmosphere. The Tsuga extracts were diluted at an initial concentration of 2 mg/ml (2× of the final concentration) in appropriate culture media and 4 sequential dilutions were tested for each cell line. Experimental controls were included for each assay: 100 μl of media to reflect the maximum growth and viability of cells and 100 ng/ml of daunorubicin to obtain an 80% cytotoxic effect. 96-well plates were incubated for 72 hours at 37° C. in a humidified 5% CO₂ atmosphere. After incubation, Alamar Blue dye was added to each well and fluorescence was read on a Spectrafluor Plus (Tecan, Durham, N.C.). All assays were done in quadruplicate.

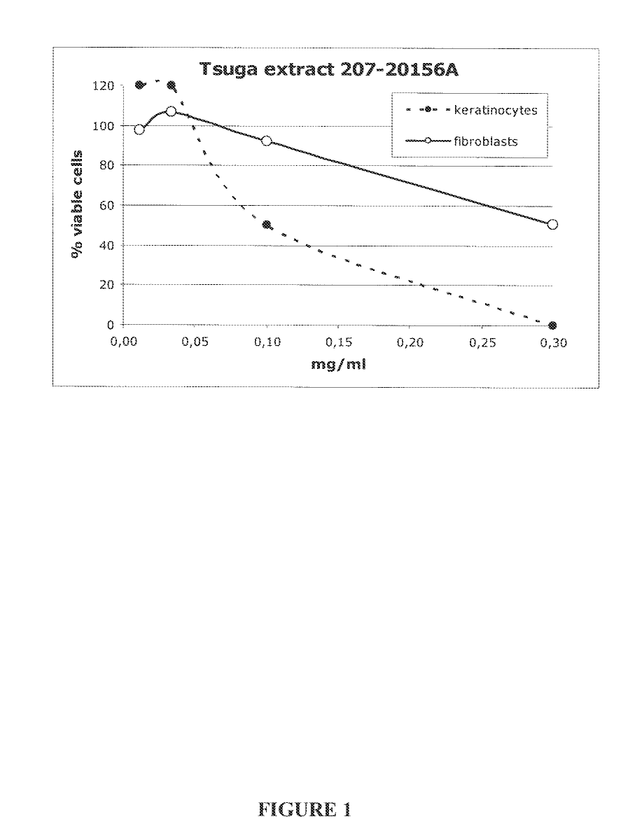

The results for Tsuga canadensis extract 207-20156A (from dried needles and small branches) are presented in FIG. 1 and are expressed as the % viable cells. The data plotted is the average of the quadruplicate results.

### Example IV

### Effect of Tsuga Extracts on Angiogenesis

60 μl Matrigel® was added to a 96-well plate flat bottom plate (Costar 3096) and incubated for 30 minutes at 37° C. in a 5% CO₂ atmosphere. A mixture of HUVECs and Tsuga extract, or a positive control (Fumagillin or GM6001) were added to each well. HUVECs were prepared as suspensions of 2.5×10⁵ cells per ml in EGM-2, then 500 μl of HUVECs preparation was mixed with 500 μl of 2× of the desired dilution of Tsuga extract or control drug and 200 μl were added to each well. Four dilutions of each extract were tested in duplicate. After 18-24 hours at 37° C. in 5% CO₂, the cells had migrated and organized into cords.

The number of cell junctions was counted in 3 randomly selected fields and the inhibition of cord formation is calculated as follows:

[(A−B)/A]×100,

where A is the average number of cell junctions per field in the control well and B is the average number of cell junctions per field in the treated wells.

Following the protocol outlined above, Tsuga canadensis extract 20156A inhibited cord formation by 85% at a concentration of 30 μg/ml, while Fumagillin at 15 μg/ml inhibited cord formation by 100% and GM6001 at 25 μM inhibited cord formation by 78%.

### Example V

### Inhibition of Contractile Force of Fibroblasts by Tsuga Extracts

The following example demonstrates the ability of Tsuga extracts prepared as described in Example I to inhibit contractile force of fibroblasts in an in vitro skin model. The skin model comprises human skin fibroblasts imbedded in a collagen I matrix and provides an in vitro representation of dermal contraction resulting from tractional forces generated by fibroblasts. Partial or permanent dermal contraction can play a role in the formation of wrinkles. Thus, extracts capable of inhibiting the contractile force of fibroblasts, have the potential to provide a dermo-decontraction anti-ageing effect in the skin.

The ability of the Tsuga extracts to inhibit the contractile force of fibroblasts was evaluated on human skin fibroblasts (Cascade Biologics, Portland, Oreg.). The cells were imbedded in a collagen I matrix to create a derm-like environment. Fibroblasts were grown in complete M106 to 80% confluence. Free-floating fibroblast-populated collagen gels were prepared in 24-well plates. 500 μl of gel contains 2.5 mg/ml of collagen 1 (rat tail, BD Biosciences, Bedford, Mass.), M106 5X, NaOH 0.7N; 1×10⁵ cells and fetal bovine serum (FBS) at 20% (Wisent, St-Bruno, QC, Canada). The mix was kept on ice until distribution. The derm-like gels were allowed to polymerize for 1 hour at 37° C. in a humidified 5% CO₂ atmosphere. After incubation, the gels were detached from the wells. Media 106 was used as negative control and GM6001 (Chemicon, Temecula, Calif.) at a concentration of 50 μM was used as positive control. The Tsuga extracts were diluted in complete media 106. FBS at a final concentration of 10% was added to each well. The plate was incubated for a maximum of 7 days at 37° C. in a humidified 5% CO₂ atmosphere. All assays were performed in duplicate. Contraction was measured beginning at day 5. Contracting gels were digitally photographed and the gel areas were calculated using ImagePro software.

Control gels treated with media alone have the smallest area and represent the contracted control. GM6001 was able to provide limited, but not complete, inhibition of contraction. Tsuga canadensis extract 20156A inhibits the tractional force of fibroblasts by 103% at a concentration of 100 μg/ml and Tsuga canadensis extract 20252-PLA inhibits the tractional force of fibroblasts by 128% at a concentration of 40 μg/ml, while GM6001 inhibits the tractional force of fibroblasts by 85% at a concentration of 50 μM.

### Example VI

### Non-Irritating Behaviour of Tsuga Extracts

This example provides results showing the non-irritating quality of the Tsuga extracts. The amount of interleukin-8 (IL-8) released in the following assay is used to quantify a possible irritating reaction after exposure of keratinocytes to the Tsuga extract.

Release of IL-8 was evaluated on human skin keratinocytes (Cascade Biologics, Portland, Oreg., catalog number C-005) and measured using the Quantikine hIL8 ELISA kit (R&D Systems, Minneapolis, Minn., catalog number D8000C). Keratinocytes were first grown in a 96-well plate using complete medium M154 (M154+HKGS cat S-001 from Cascade Biologics). This medium was used as a negative (non-irritating) control, while 2.5 μM phorbol 12-myristate 13-acetate (PMA) (Sigma-Aldrich Canada, Oakville, Ontario) was used as a positive (irritating) control. All extracts and controls were diluted in complete medium M154. Cells were seeded into 96-well plates at a concentration of 8×10³ cells/well in complete M154 medium and plates were incubated for 48 hours at 37° C. in a humidified 5% CO₂ atmosphere. The medium was removed and 200 μl of Tsuga extract or control were added to the wells (all performed in duplicate) and then the plates were incubated for 48 hours at 37° C. in a humidified 5% CO₂ atmosphere. ELISA was performed using following the manufacturer's recommendations (R&D Systems). The absorbance was read at 450 nm on the Spectrafluor Plus plate reader (Tecan).

Controls treated with M154 medium showed the lowest IL-8 release and this amount was taken as the minimum IL-8 release. PMA induced a strong inflammatory response, which was taken as the highest irritating level (highest IL-8). The evaluation of cytokine release stimulated by a Tsuga extract in this experiment enables a maximum concentration of the extract to be set for further in vitro studies.

Following the above protocol, Tsuga canadensis extract 20252-PLA stimulated 2.2 pg/ml of IL-8 release at a concentration of 200 μg/ml, while PMA stimulated 282 pg/ml of IL-8 release at a concentration of 5 μM. The basal level was evaluated at 18 pg/ml.

### Example VII

### Inhibition of UV-Induced Proteolytic Activity

This example demonstrates the potential of the Tsuga extracts to protect the skin from proteolytic damage after sun exposure.

Fibroblasts were first grown in 24-well plates using complete M106 medium (M06+LSGS from Cascade Biologics) at a concentration of 6×10⁴ cells/500 μl/well. The plates were incubated 48 hours at 37° C. in a humidified 5% CO₂ atmosphere. The medium was removed and the cells were washed 2 times with HBSS. After complete removal of liquid, the cells were irradiated with 25 J/cm² of UVA light. Tsuga extracts and controls were added at 500 μl/well. The medium was used as a negative (no UV protection) control, while 50 μM GM6001 was used as a positive control. All extracts and controls were diluted in complete M106 medium. All assays were done in duplicate except for controls that were done in quadruplicate. Plates were incubated for 24 hours at 37° C. in a humidified 5% CO₂ atmosphere. Supernatant from each well was assayed or kept at −80° C. until use.

The supernatants were assayed for their overall proteolytic activity using the internally quenched peptide. MCA-Pro-Leu-Gly-Dpa-Ala-Arg-NH₂ (Bachem, Torrance, Calif.). Supernatants were distributed in a 96-well black plate, with or without peptide. The reaction took place in a 50 mM Tris, 150M NaCl, 5 mM CaCl₂, 0.5 mM ZnCl₂ pH 7.5 buffer. The incubation was at 37° C. Each of the measurements was done in duplicate. Readings were taken, at 20, 40 and 60 minutes, on the Polarion plate reader (Tecan) with excitation at 330 nm and emission at 400 nm.

The UVA-irradiated cells showed a higher proteolytic activity than the non-irradiated cells. Inhibition of this enzymatic cleavage could protect against skin damage such as photoaging. Controls treated with medium alone represent the highest proteolytic activity. GM6001 was used as positive control.

Following the above protocol, Tsuga canadensis extract 20156A showed good inhibition of the overall proteolytic cleavage. An inhibition of 41% at a concentration of 50 μg/ml was evaluated for this extract.

### Example VIII

### Anti-Inflammatory Effect of Tsuga Extracts on UVA-Induced Inflammation in Keratinocytes

This example provides results showing the anti-inflammatory effect of Tsuga extracts when inflammation is induced in keratinocytes. The amount of interleukin-1 alpha (IL-1) released is used to quantify the inflammatory reaction after exposure of the keratinocytes to UVA light irradiation.

IL-1 release was evaluated in human skin keratinocytes (Cascade Biologics, Portland, Oreg.) and was measured using the Quantikine hIL1 ELISA kit (R&D Systems, Minneapolis, Minn.). Keratinocytes were first grown in a 96-well plate using complete M154 medium with different concentrations of extracts for 48 hours at 37° C. in a humidified 5% CO₂ atmosphere. All extracts and controls were diluted in complete M154 medium. Cells were rinsed with HBSS and irradiated with UVA at 20 J/cm². HBSS was removed and complete M154 medium containing controls or extracts was added to respective wells. Plates were incubated for another 24 hours.

ELISA was performed following the manufacturer's recommendations (R&D Systems). The absorbance was read at 450 nm on the Spectrafluor Plus plate reader (Tecan).

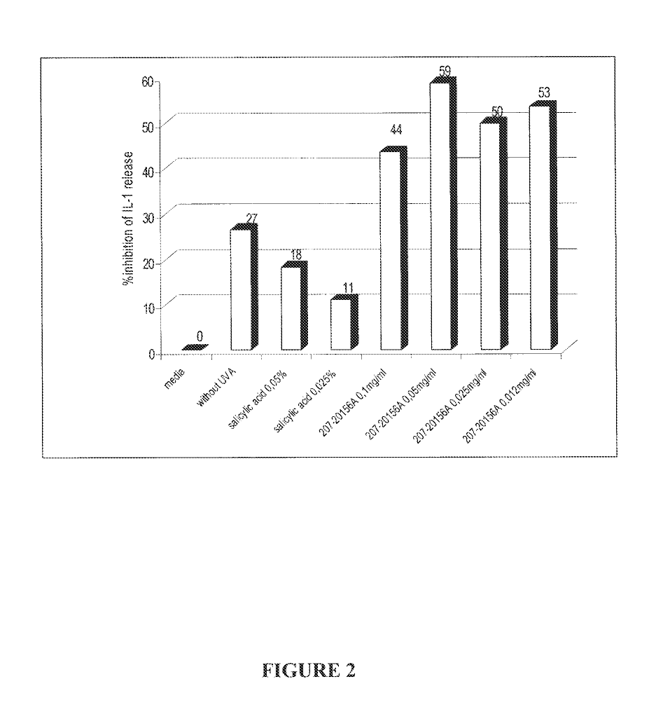

Controls treated with M154 medium showed the highest inflammatory response and this amount was taken as the highest IL-1 release. Controls in medium without UVA irradiation showed the lowest IL-1 release. The inhibition of IL-1 release is proportional to the anti-inflammatory effect. The inhibition of inflammation by Tsuga canadensis extract 207-20156A according to this protocol is shown in FIG. 2.

As shown in FIG. 2, even at 0.012 mg/mL Tsuga canadensis extract 207-20156A inhibited IL-1 release by 53%, whereas salicylic acid, which is a well-known anti-inflammatory agent, inhibited IL-1 release by 11% at 0.025% and by 27% at 0.05%.

### Example IX

### Effect of Tsuga Extracts on the Expression of Psoriasis Markers in Keratinocytes

This Example provides results showing the effect of Tsuga extracts on the expression of different markers expressed by psoriasis-like keratinocytes. Cell markers such as cytokeratine-16 (CK16), CK10, transglutaminase II, involucrin and SKALP are over-expressed in keratinocytes that are involved in psoriasis. Induction of cornification and differentiation in keratinocytes results in over-expression of these markers, mimicking the psoriatic state. The Tsuga canadensis extract used in this Example (Tsuga canadensis extract 9338 4AC022C4) was an alcoholic extract prepared by extracting 10 g of dried plant material from Tsuga canadensis lot 9338 with 100 ml of Ethanol:MeOH 85:15, following the general protocol outlined in Example I.

Human skin keratinocytes (Cascade Biologics, Portland, Oreg.) were first grown in 96-well plates using complete M154 medium with various concentrations of Tsuga canadensis extract 9338 for 72 hours at 37° C. in a humidified 5% CO₂ atmosphere. Differentiation was initiated with 10% fetal calf serum added to the well at the start of the incubation period. The cells were rinsed with HBSS and fixed with a 70:30 (ethanol:acetone) solution. After rinsing with PBS, 4% dried milk in PBS was added to each well and the plate incubated for 1 hour at 37° C. Each of the antibodies anti-CK 10, anti-CK16, anti-involucrin, anti-SKALP and anti-transglutaminase II was diluted, as recommended by the manufacturer, in PBS containing 1% dried milk, added to the wells and the plate incubated for 1 hour at 37° C. Four washes were performed with PBS-0.05% Tween prior to the addition of the secondary antibody: anti-IgG labelled with Alexa 594 or Alexa 485 (Molecular Probes) diluted in PBS with 1% dried milk to the recommended concentration. The plate was then incubated for an additional 1 hour at 37° C. Four washes were then performed prior to evaluation under a fluorescent microscope. The results are shown in Table I and show that the effect of Tsuga canadensis extract 9338 4AC022C4 on the expression of the markers was comparable to that of the positive control Dithranol, a known anti-psoriasis agent.

TABLE I
Evaluation of the Effect of Tsuga canadensis extract 9338 4AC022C4 on
Expression of Psoriasis Markers

| | | | | | | |
|---|---|---|---|---|---|---|
| No | − | +++ | +++ | +++ | +++ | +++ |
| induction | | | | | | |
| Induced | − | − | − | − | − | − |
| cells | | | | | | |
| Dithranol | 4.5 μg/ml | +/− | + | ++ | +/− | + |
| Tsuga | 200 μg/ml | +/− | ++ | ++ | + | + |
| 9338 | | | | | | |
| 4AC022C4 | | | | | | |

### Example X

### Anti-Inflammatory Effect of Tsuga Extracts in Humans: a Case Report

### Materials and Methods

Four creams were prepared using a 50:50 butylene glycol:water Tsuga canadensis extract prepared as described in Example I. The creams were as follows (all % are w/w):

Cream #1: 0.5% retinol
Cream #2: 0.5% retinol and 5% Tsuga canadensis extract
Cream #3: 1% retinol
Cream #4: 1% retinol and 5% Tsuga canadensis extract

The patient was a healthy female volunteer age 39. She applied all four creams (#1 to #4, above) twice a day in a blinded manner to her forearms. She noted any sign of irritation. The study was stopped when the redness due to irritation was too strong and discomfort appeared.

### Results

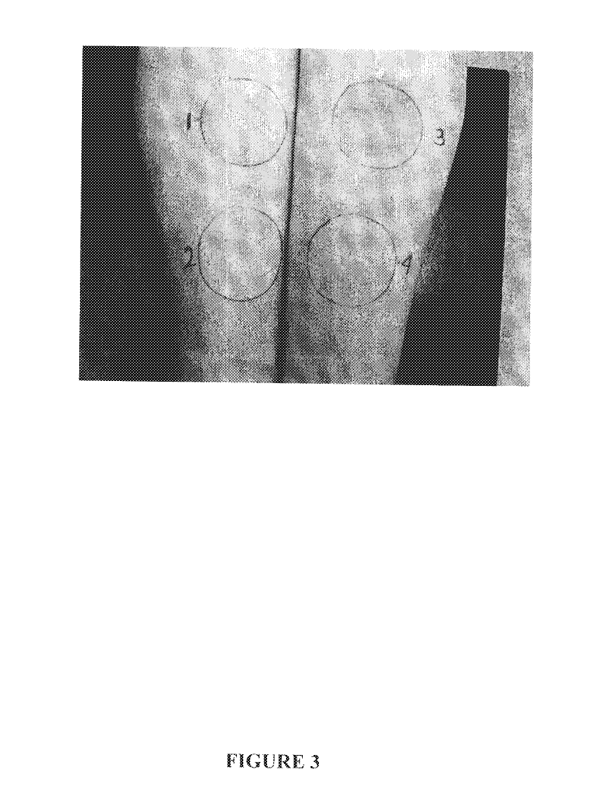

The results after 21 days of treatment are shown in FIG. 3. An irritating effect of retinol at 1% (Cream #3) was observed starting at day 10 of the experiment. The redness was intense at day 18 to 21, when the patient was told to stop the study. The Tsuga extract at 5% (Cream #4) clearly helped to diminish the level of inflammation caused by retinol from an assessment of “3” to an assessment of “1” based on qualitative evaluation of the redness of the skin. The intensity of inflammation was less when the 0.5% retinol cream (Cream #1) was used. However, addition of the Tsuga extract at 5% (Cream #2) also helped to diminish the level of inflammation caused by this lower amount of retinol.

### Example XI

### Determination of Irritation or Sensitization by Tsuga Extracts on Hunan Skin I

This example and Example XIV below demonstrate that the Tsuga extracts are non-irritating to human skin. The test employed was the HRIPT (Human Repeat Insult Patch Test), which determines if a topical agent has the potential to induce irritation or sensitization of any kind.

52 volunteers were selected for the study. The volunteers were men and women of ages 23 to 57. The agent, Tsuga canadensis extract 20252-PLA 5% (v/v) in petroleum jelly, was applied to the skin of the volunteers repeatedly using 10 patches over a period of 3 weeks. The patches used in the study were TruMed® semi-occlusive, cotton “Nova® net embossed” with “Avery® 4750U tape” adhesive backing. After a rest period (incubation phase) varying from 10 to 14 days, a challenge phase was conducted. The patch was applied for 48 hours and removed. The test sites were cleaned and examined for any signs of intolerance or irritation by a dermatologist.

### Results:

Under the conditions of the study, Tsuga canadensis extract 20252-PLA produced no signs of cutaneous irritation or skin sensitization in either the induction phase or the challenge phase of the test. The extract is therefore considered non-irritant and potentially hypo-allergenic. In addition, given the control provided by a dermatologist, the test product may bear the claim “Tested under control of a dermatologist.”

### Example XII

### Anti-Microbial Activity of Tsuga Extracts I

To determine the antimicrobial effect of Tsuga extracts, Escherichia coli (ATCC 8739), Staphylococcus aureus (ATCC 6538) and Pseudomonas aeruginosa (ATCC 9027) were used to challenge different concentrations of Tsuga canadensis extract 20156A (diluted in water). E. coli was inoculated at 1.5×10⁶ bacteria/ml in 10 ml of diluted extract; S. aureus was inoculated at 2×10⁶ bacteria/ml in 10 ml of diluted extract and P. aeruginosa was inoculated at 1×10⁶ bacteria/ml in 10 ml of diluted extract. All samples of extract or vehicle (50:50 butylene glycol:water) were incubated at 37° C. and evaluated at 0, 7, 14 and 28 days. The effectiveness was analyzed by determining the colony forming unit (CFU) per g on tryptic soy agar.

The results are summarized in Tables II A, B and C and demonstrate that when the Tsuga extract was challenged with 3 different species of bacteria, a significant inhibition of growth of the bacteria resulted even after the first incubation period.

TABLE II
Evaluation of the antimicrobial effect of Tsuga canadensis
extract 20156A against E. coli, S. aureus and
P. aeruginosa (results in CFU)
A. E. coli

| | | | | |
|---|---|---|---|---|
| Tsuga extract 20156A | 5.1E+03 | 180 | 0 | 0 |
| 100% | | | | |
| Tsuga extract 20156A | 2.2E+05 | 100 | 0 | 0 |
| 50% | | | | |
| Tsuga extract 20156A | 3.6E+05 | 50 | 0 | 0 |
| 25% | | | | |
| Tsuga extract 20156A | 3.7E+05 | 5.3E+03 | 0 | 0 |
| 10% | | | | |
| Vehicle 100% | 1.7E+05 | 0 | 0 | 0 |
| Vehicle 50% | 3.5E+05 | 50 | 0 | 0 |
| Vehicle 25% | 3.6E+05 | 6.1E+05 | 9.0E+04 | 0 |
| Vehicle 10% | 3.2E+05 | 9.0E+06 | 7.7E+08 | 1.2E+09 |
| Bacteria + Nutrient Broth | 3.3E+05 | 7.7E+12 | 5.9E+15 | 2.0E+14 |

B. S. aureus

| | | | | |
|---|---|---|---|---|
| Tsuga extract 20156A | 0 | 0 | 0 | 0 |
| 100% | | | | |
| Tsuga extract 20156A | 0 | 0 | 0 | 0 |
| 50% | | | | |
| Tsuga extract 20156A | 0 | 0 | 0 | 0 |
| 25% | | | | |
| Tsuga extract 20156A | 15 | 0 | 0 | 0 |
| 10% | | | | |
| Vehicle 100% | 4.8E+02 | 0 | 0 | 0 |
| Vehicle 50% | 5.5E+04 | 0 | 0 | 0 |
| Vehicle 25% | 4.5E+04 | 4.2E+06 | 1.7E+07 | 2.4E+06 |
| Vehicle 10% | 8.1E+04 | 3.7E+06 | 9.6E+07 | 9.0E+05 |
| Bacteria + Nutrient Broth | 4.8E+04 | 2.5E+13 | 5.0E+11 | 2.6E+14 |

C. P. aeruginosa

| | | | | |
|---|---|---|---|---|
| Tsuga extract 20156A | 4.5E+03 | 0 | 0 | 0 |
| 100% | | | | |
| Tsuga extract 20156A | 1.0E+03 | 0 | 0 | 0 |
| 50% | | | | |
| Tsuga extract 20156A | 5.5E+03 | 0 | 0 | 0 |
| 25% | | | | |
| Tsuga extract 20156A | 1.1E+04 | 50 | 0 | 0 |
| 10% | | | | |
| Vehicle 100% | 2.0E+03 | 0 | 0 | 0 |
| Vehicle 50% | 1.4E+04 | 0 | 0 | 0 |
| Vehicle 25% | 7.5E+03 | 3.1E+02 | 0 | 0 |
| Vehicle 10% | 8.0E+03 | 3.9E+04 | 1.4E+04 | 0 |
| Bacteria + Nutrient Broth | 1.1E+04 | 9.4E+12 | 5.1E+12 | 5.3E+14 |

¹ All % are v/v.

### Example XIII

### Exploratory Study of the Anti-Psoriatic Effect of a Dermatological Formulation of a Tsuga Extract

Four volunteers were selected for the study. The volunteers were men and women of ages over 18 with mild to moderate psoriatic lesions (plaques). The agent, Tsuga canadensis extract 5% (v/v) formulated in a cream (Lotion Glaxal base from Wellskin), was applied onto the plaque twice a day for a period of 8 weeks. A placebo was applied to a similar plaque. The patients received two bottles in a blinded manner and were required to fill out a follow-up sheet. Patients had a follow-up evaluation by a dermatologists at weeks (W) 0, 4 and 8, in which the lesions were scored for erythema (E), squame or dryness (S), thickness or induration (I) and surface (A). The results are presented in Tables IIIA and B below.

TABLE III
Evaluation of Psoriatic Lesions Treated with
5% Tsuga canadensis Extract after 8 Weeks

| | | | | | |
|---|---|---|---|---|---|
| Lesion or plaque | W 0 | W 4 | W 8 | ΔW 4 − W 0 | ΔW 8 − W 0 |

A. Tsuga canadensis Extract

| | | | | | |
|---|---|---|---|---|---|
| Squames (S) | 12 | 8 | 7 | −33% | |
| Erythema (E) | 13 | 11 | 10 | −15% | |
| Thickness (I) | 12 | 10 | 9 | −17% | −25% |
| Surface (A) | 9 | 9 | 8 | 0% | −11% |
| Total | 46 | 38 | 34 | −17% | −26% |

B. Placebo

| | | | | | |
|---|---|---|---|---|---|
| Squames (S) | 13 | 9 | 8 | −31% | −38% |
| Erythema (E) | 13 | 12 | 11 | −8% | −15% |
| Thickness (I) | 12 | 9 | 9 | −25% | −25% |
| Surface (A) | 13 | 13 | 12 | 0% | −8% |
| Total | 51 | 43 | 40 | −16% | −22% |

Under the conditions of the study, the Tsuga canadensis extract at 5% (v/v) in a dermatological formulation produced a beneficial effect on psoriatic lesions. The effect was primarily on two parameters: erythema (E, redness) and squame (S). The results are significant enough to warrant further clinical studies.

### Example XIV

### Determination of Irritation or Sensitization by Tsuga Extracts on Human Skin II

54 volunteers were selected for the study. The volunteers were men and women of ages 20 to 53. The agent, Tsuga canadensis extract 20376 1NG23ZB 10% (v/v) in a base cream, was applied to the skin of the volunteers repeatedly using 10 patches over a period of 3 weeks. The patches used in the study were TruMed® semi-occlusive, cotton “BBA149-129 Absorbent” with “3M1530 tape” adhesive backing. After a rest period (incubation phase) varying from 10 to 14 days, a challenge phase was conducted. The patch was applied for 48 hours and removed. The test sites were cleaned and examined for any signs of intolerance or irritation by a dermatologist.

### Results:

Under the conditions of the study, Tsuga canadensis extract 20376 1NG23ZB 10% (v/v) produced no signs of cutaneous irritation or skin sensitization in either the induction phase or the challenge phase of the test. The extract is therefore considered non-irritant and potentially hypo-allergenic. In addition, given the control provided by a dermatologist, the test product may bear the claim “Tested under control of a dermatologist.”

### Example XV

### Anti-microbial Activity of Tsuga Extracts II

To determine the antimicrobial effect of Tsuga extracts, Escherichia coli (ATCC 8739), Staphylococcus aureus (ATCC 6538) and Pseudomonas aeruginosa (ATCC 9027) were used to challenge different concentrations of Tsuga canadensis extract 20010 1NG13B (diluted in water). E. coli was inoculated at 1.5×10⁶ bacteria/ml in 10 ml of diluted extract; S. aureus was inoculated at 2×10⁶ bacteria/ml in 10 ml of diluted extract and P. aeruginosa was inoculated at 1×10⁶ bacteria/ml in 10 ml of diluted extract. All samples of extract or vehicle (50:50 butylene glycol:water) were incubated at 37° C. and evaluated at 0, 7, 14 and 28 days. The effectiveness was analyzed by determining the colony forming unit (CFU) per g on tryptic soy agar.

The results are summarized in Tables IV A, B and C and demonstrate that when the Tsuga extract was challenged with 3 different species of bacteria, it showed a significant inhibition of growth even after the first incubation period.

TABLE IV
Evaluation of the antimicrobial effect of Tsuga canadensis
extract 20010 1NG13B against E. coli, S. aureus and
P. aeruginosa (results in CFU)
A. E. coli

| | | | | |
|---|---|---|---|---|
| Tsuga extract 20010 | 2.44E+05 | 0 | 0 | 0 |
| 1NG13B 10% | | | | |
| Tsuga extract 20010 | 3.29E+05 | 0 | 0 | 0 |
| 1NG13B 5% | | | | |
| Tsuga extract 20010 | 2.81E+05 | 0 | 0 | 0 |
| 1NG13B 1% | | | | |
| Vehicle 10% | 2.78E+05 | 0 | 0 | 0 |
| Vehicle 5% | 2.95E+05 | 5.56E+07 | 3.40E+09 | 5.60E+05 |
| Vehicle 1% | 3.72E+05 | 6.21E+07 | 4.20E+09 | 7.60E+07 |
| Bacteria + Nutrient Broth | 3.27E+05 | 3.96E+11 | 4.50E+14 | 1.29E+17 |

B. S. aureus

| | | | | |
|---|---|---|---|---|
| Tsuga extract 20010 | 1.00E+04 | 0 | 0 | 0 |
| 1NG13B 10% | | | | |
| Tsuga extract 20010 | 9.10E+04 | 0 | 0 | 0 |
| 1NG13B 5% | | | | |
| Tsuga extract 20010 | 1.38E+05 | 7.75E+07 | 4.60E+09 | 2.40E+07 |
| 1NG13B 1% | | | | |
| Vehicle 10% | 1.35E+05 | 5.20E+06 | 7.00E+08 | 5.00E+07 |
| Vehicle 5% | 1.10E+05 | 2.85E+07 | 6.00E+08 | 3.00E+07 |
| Vehicle 1% | 1.30E+05 | 2.77E+07 | 6.00E+08 | 2.00E+10 |
| Bacteria + Nutrient Broth | 1.47E+05 | 8.01E+11 | 9.08E+15 | 2.26E+17 |

C. P. aeruginosa

| | | | | |
|---|---|---|---|---|
| Tsuga extract 20010 | 1.88E+05 | 2.00E+01 | 0 | 0 |
| 1NG13B 10% | | | | |
| Tsuga extract 20010 | 1.26E+05 | 6.83E+07 | 2.25E+10 | 2.94E+09 |
| 1NG13B 5% | | | | |
| Tsuga extract 20010 | 1.14E+05 | 6.83E+07 | 7.35E+10 | 2.34E+09 |
| 1NG13B 1% | | | | |
| Vehicle 10% | 1.29E+05 | 4.02E+07 | 7.40E+09 | 1.92E+09 |
| Vehicle 5% | 1.49E+05 | 7.87E+07 | 2.32E+10 | 1.22E+09 |
| Vehicle 1% | 8.70E+04 | 7.10E+07 | 6.74E+10 | 1.25E+09 |
| Bacteria + Nutrient Broth | 1.54E+05 | 5.52E+11 | 4.35E+15 | 2.00E+17 |

¹ All % are v/v.

### Example XVI

### Exemplary Dermatological Formulations Comprising a Tsuga Extract

The following formulation comprising a Tsuga canadensis extract is a non-limiting example of a suitable formulation for human efficacy studies (such as those described in Examples XVII and XVIII). The formulation can be modified for commercial use as is known in the art. All % are w/w.

Cream:

| | |
|---|---|
| Beeswax | 1-2% |
| Glycerin | 2-4% |
| Xanthan Gum | 0.5-1% |
| PPG-15 stearyl ether | 6-10% |
| Stearic acid | 1-2% |
| Kaleol 8670 | 1-4% |
| EDTA | 0.25-0.5% |
| Shea butter | 1-3% |
| Extract 20252 | 0.1-5% |
| Preservatives | 0.2-1% |
| Water | q.s. 100% |

Other examples of formulations comprising Tsuga canadensis extracts include those in which the extract is added in an amount between 0.1 and 10% (w/w) into a pre-made skin formulation. For example, a cream base, a lotion, an aftershave lotion, a soothing mask, or petrolatum jelly.

### Example XVII

### Evaluation of Wrinkle Attenuation by the Tsuga Extracts

The following Example provides an exemplary protocol that may be followed to evaluate the ability of a Tsuga extract to improve the appearance of wrinkles in human volunteers.

Healthy volunteers are divided in 2 groups. Each group of volunteers receives a composition containing 5% (v/v) Tsuga extract. The composition is applied around the eyes twice a day for a period of 12 weeks. Each volunteer is asked to comment on the composition and the improvement they feel in their wrinkles with a scale from −3 (greatly worsened) to 0 (no change) to +3 (greatly improved).

The improvement in the appearance of the wrinkles can also be determined by profilometry. In this case, replicas (negatives of the skin surface) of the eye contour zones of the volunteers are obtained prior to and after treatment by applying silicone polymer onto the “crow's feet” of the eye contour zone, while the volunteer maintains an upright but sitting position. Replicas of the crow's feet are then analyzed by a computerized digital image processing system coupled to Quantirides® software to obtain the skin's topography. This standard technique is based on measuring the shadows cast when an incident light is inclined at an angle of 35° on the replica.

The analyzed parameters are: the total area of wrinkled skin, the number and the mean depth of the depressions due to the cutaneous relief, and depth of deep and medium-deep wrinkles. The wrinkles are differentiated by classes of depth (Class 1 for 0-55 mm: Class 2 for 55-110 mm; and Class 3 for 110-800 mm).

### Example XVIII

### Evaluation of the Anti-Inflammatory Activity of the Tsuga Extracts in Humans

The following example provides an exemplary protocol for assessing the ability of the Tsuga extracts to attenuate UV-induced erythema.

The MED test measures the minimum UV dose required to produce a distinct reaction in the form of redness that appears 24 hours after exposure. The MED of all volunteers must be measured prior to any treatment that attempts to attenuate the skin response to UV-induced erythema. Accordingly, at Day D0, the skin color for each volunteer is measured in the test zone (forearm) using a Mexameter® to approximate the MED of the individual volunteer. Actinic erythema is induced using a UV simulator to the selected zone by a series of 5 consecutive increasing irradiations on a non-treated site on the right forearm and on a site treated with the extract on the left forearm. The skin response on each forearm is then assessed 24 hrs later (Day D1) to calculate the individual MED (the lowest dose to which a distinct reaction is observed). The change observed with the extract can be scored, for example, on a numerical scale in which 5 indicates “very improved” and 0 indicates “no change.” The ability of the extract to decrease the observed redness is indicative of its anti-inflammatory effect.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1-75. (canceled)

76. A method for ameliorating a skin inflammation in a subject in need thereof, comprising:
topically administering to said subject an effective amount of a glycolic extract from a Tsuga canadensis, or one more active ingredients isolated therefrom.

77. The method of claim 76, wherein said skin inflammation is associated with acne.

78. The method of claim 76, wherein the extract is incorporated into a dermatological formulation.

79. The method of claim 78, wherein the dermatological formulation is formulated for topical administration.

80. The method of claim 79, wherein the dermatological formulation is compatible with a mammalian skin.

81. The method of claim 80, wherein the mammalian skin is a human skin.

82. The method of claim 78, wherein the dermatological formulation:
is selected from the group consisting of. a cosmetic; an anti-ageing product; and, a formulation for sensitive skin; or,
the dermatological formulation is formulated as a cosmetic; an anti-ageing product; or, a formulation for sensitive skin.

83. The method of claim 76, wherein the extract is prepared by glycolic extraction of needles, twigs, small branches or a combination thereof, from the plant Tsuga canadensis.

84. The method of claim 83, wherein the extract is prepared by extraction of needles from the plant.

85. The method of claim 76, wherein the glycolic solvent comprises a butylene glycol or a mixture of a butylene glycol and water.

86. The method of claim 85, wherein the glycolic solvent is butylene glycol (100%).

87. The method of claim 85, wherein the glycolic solvent comprises a combination of butylene glycol and water, wherein the range of butylene glycol. water is from between about 100:0 to about 20:80 (v/v).

88. The method of claim 85, wherein the glycolic solvent is selected from the group consisting of. butylene glycol (100%); butylene glycol/water 50:50 (v/v); and, butylene glycol/water 20:80 (v/v).

89. The method of claim 76, wherein the Tsuga canadensis extract is prepared by a process comprising. extracting a plant material with a glycolic solvent for a time period between about 4 hours and about 48 hours, and at a temperature of between about 4° C. to about 32° C.

90. The method of claim 78, wherein the Tsuga extract present in the dermatological formulation is in the range of between about 0.1% (v/v) to about 20% (v/v).
